(19) United States
(12) Patent Application Publication
(10) Pub. No.: US 2003/0044412 A1
(43) Pub. Date: Mar. 6, 2003

(54) Membrane estrogen receptor-directed therapy in breast cancer

(76) Inventors: Richard J. Pietras, Sherman Oaks, CA (US); Diana C. Marquez-Garban, Los Angeles, CA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 10/204,920

(22) PCT Filed: Feb. 23, 2001
(86) PCT No.: PCT/US01/05897

(51) Int. Cl. A61K 39/395

(52) U.S. Cl. 424/144.1; 424/143.1

(57) ABSTRACT

Methods of diagnosing and treating mammalian tumors with molecules including anti-estrogen receptor immunoglobulin polypeptides are provided. In an illustrative embodiment, anti-estrogen receptor immunoglobulin polypeptides specific to distinct epitopes of the ligand-binding domain of estrogen receptor are contacted with membrane-associated estrogen receptor under conditions which allow binding of the anti-estrogen receptor immunoglobulin polypeptide to a degree sufficient to inhibit tumor growth by inhibiting the activation of the membrane-associated estrogen receptor. Injectable compositions for treating certain mammalian tumors with monoclonal antibodies and methods for diagnosing mammalian cancers which express an estrogen receptor associated with the surface membrane of the cells are also disclosed. Further, alternate methods for blocking intracellular signal transduction emanating from the activation of membrane-associated estrogen receptor forms are also presented. These approaches also appear sufficient to inhibit tumor growth.

### CROSS REFERENCE TO RELATED APPLICATIONS

This application claims the benefit of U.S. provisional patent application No. 60/185,026, filed Feb. 25, 2000, the entire contents of which are incorporated herein by reference.

### FIELD OF THE INVENTION

The present invention is directed to methods of inhibiting the signalling of a membrane-associated estrogen receptor and diagnoses and treatments for mammalian pathologies.

### BACKGROUND OF THE INVENTION

Cancers of the breast are one of the leading causes of death among women, with the cumulative lifetime risk of a woman developing breast cancer estimated to be 1 in 8. Consequently, the identification of new therapeutic modalities is of significant interest to health care professionals.

Breast cancer arises from the uncontrolled division of breast cells which can spread into and destroy normal tissues. Growth of breast cells is normally regulated by hormones such as estrogen which bind to specific receptors that are present in more than two-thirds of breast cancers. In the traditional understanding of estrogen action, these hormone receptors localize in the nucleus and are activated by binding estrogen and subsequently, with DNA in order to communicate growth-promoting signals to specific genes in the DNA of the cell. New findings also provide evidence that changes in cancer-related genes, such as those coding for growth factor receptors, may lead to the emergence of estrogen-independent activation of hormone receptors. Specifically, the events leading to the activation of the estrogen receptor signal are associated with disregulated cell division.

New approaches to cancer therapy involve efforts to cut the communication lines between hormone receptors and the cell nucleus, thus slowing or blocking cell division. Antiestrogen therapy is one well-known example of these approaches, and it is often used to treat breast cancer and to prevent the recurrence of disease. Unfortunately, many patients do not respond to this therapy, and most treated patients eventually become resistant to antiestrogens. In addition, antiestrogens that are now available can result in abnormal uterine growth and thromboembolic events. The failure of antihormone therapy in the clinic appears to be due to many factors, including the emergence of estrogen-independent growth that is no longer responsive to treatment with antiestrogen agonists.

New options for antiestrogen treatment are clearly needed, and alternative therapies may now derive from findings showing that a portion of estrogen receptor molecules are associated with the plasma membranes of human breast cancer cells. Specifically, while a number of studies have implicated the existence of membrane-associated estrogen receptors, these observations were incompatible with prevailing dogma that the estrogen receptor was a gene-activating factor present only in the nucleus of the cell. Recently, however, evidence for the presence of one or more membrane-associated estrogen receptors has been accumulating (see ref. 64: Ann. Rev. Physiol., 59: 365, 1997), providing evidence that the classical model of estrogen action is incomplete and must be expanded to include the estrogen receptor as a component of other cell signaling pathways (see ref. 40: Proc. Natl. Acad. Sci. USA, 96: 4686, 1999).

As estrogen receptor molecules occur in association with plasma membranes, these molecules may interact with other molecules involved in oncogenesis such as transmembrane erb B growth factor receptors. In particular, it is known that expression of HER-2/erb-B2 receptors occurs in many human breast cancers, and the enzyme activity of HER-2 may play a role in the activation of estrogen receptor even in the absence of estrogen. As recognized in recent reviews (see ref. 29: Nature Medicine, 4: 761, 1998), if active cross-communication between estrogen receptor and HER-2 growth factor receptor occurs and leads to promotion of cancer growth, this signaling complex may offer an important new target for therapeutic intervention. Moreover, as the overexpression of the HER-2 receptor in human breast cancers is associated with the failure of antiestrogen therapy in the clinic, understanding the biological basis of the association between membrane-associated estrogen receptors and HER-2 receptors may also help to improve decisions on patient management and to increase patient survival.

With the significant cumulative lifetime risk of a woman developing breast cancer, there is an urgent need to develop both prophylactic and therapeutic methods of treatment that are more effective, less invasive and accompanied by fewer side effects than existing methods. In spite of the recent discovery of the heritable breast cancer susceptibility loci including BRCA1 and other genes (see e.g. Miki et al., Science 266:66-71 (1994)), and the increasing ability of physicians to identify women with elevated breast cancer risk, the majority of prophylactic methods are limited to physical monitoring and prophylactic mastectomy. Although biologically-based therapies in the form of antiestrogens have been a mainstay in breast cancer treatment to date, studies are needed to generate novel therapeutic strategies directed at membrane estrogen receptors and their interaction with erb B/HER receptors and other intracellular signaling pathways may also yield novel agents for use in the clinic.

In view of the above, what is needed in the art is the identification of novel methods which aid in the prevention and treatment of cancers such as cancers of the mammary gland. In this context, optimal methods are those which have a wide application both in the diagnosis of cancer, as well as the prophylactic and therapeutic treatment of cancer.

### SUMMARY OF THE INVENTION

The present invention is directed to methods of inhibiting the signalling of a membrane-associated estrogen receptor by exposing the membrane-associated estrogen receptor to an inhibitory molecule that inhibits membrane-associated estrogen receptor signalling. Illustrative embodiments of the invention provided herein comprise methods of diagnosing and/or treating an individual suspected of suffering from a cancer which expresses membrane-associated estrogen receptor comprising the steps of administering to said individual a pharmaceutical composition comprising a pharmaceutically acceptable carrier or diluent, and a diagnostically or therapeutically effective amount of a compound consisting of an inhibitory ligand such as an anti-membrane-associated estrogen receptor immunoglobulin polypeptide. In addition to these immunoglobulin polypeptides, the antitumor efficacy of two different synthetic inhibitors of MAP kinase-activating enzymes (PD 98059 and UO126) in estrogen-stimulated human breast cancer cells is also disclosed. Both synthetic inhibitors elicit the blockade of estrogen-induced MAP kinase activation in breast cancer cells. This effect of the inhibitors leads to blockade of estrogen-induced cell proliferation. Similarly, the antiestrogen, ICI 182780, can block cell proliferation stimulated by the membrane-associated estrogen receptor. Inhibition of phosphoinositol-3 kinase (PI3K) by administration of LY294002 also interferes with estrogen-stimulated activation of Akt kinase, an effector immediately downstream of PI3K that may promote enhanced cell survival. This data demonstrates that alternative approaches can be used to inhibit the proliferative activity of signals generated by activation of the membrane-associated estrogen receptor. Consequently, the disclosure provided herein teaches that membrane-associated estrogen receptor activity can be inhibited by anti-membrane estrogen receptor antibodies, antiestrogens (ICI 182,780) and inhibitors of signaling pathways, such as MAP kinase and PI3/Akt kinase, that originate at the surface membrane.

The methods disclosed herein include a number of different embodiments. In an illustrative embodiment, the invention consists of a method for inhibiting the growth of a breast cancer cell which expresses the estrogen receptor associated with its cell membrane by contacting the cell with an amount of anti-estrogen receptor immunoglobulin polypeptide sufficient to inhibit cell growth. In preferred embodiments of this method, the anti-estrogen receptor immunoglobulin polypeptide recognizes and binds the ligand binding domain of the estrogen receptor. In preferred embodiments of this method, the anti-estrogen receptor immunoglobulin polypeptide is selected from the group consisting of monoclonal antibodies Ab 1 and Ab 2.

A related embodiment of the invention consists of a method for treating a mammalian cancer cell which expresses estrogen receptor associated with its cell membrane by contacting the cancer cell with the anti-estrogen receptor immunoglobulin polypeptide under conditions which allow the anti-estrogen receptor immunoglobulin polypeptide to bind to the estrogen receptor associated with the surface membrane of the cancer cell to a degree sufficient to inhibit the growth of the cancer cell. In preferred embodiments of this method, the anti-estrogen receptor immunoglobulin polypeptide is selected from the group consisting of monoclonal antibodies Ab 1 and Ab 2.

Yet another related embodiment of the invention consists of a method of inhibiting the growth of a breast cancer cell having disregulated cell growth comprising the steps of confirming the presence of a estrogen receptor in the membrane of the breast cancer cell, providing a estrogen receptor immunoglobulin polypeptide specific for an epitope within the ligand binding domain of the estrogen receptor, the anti-estrogen receptor immunoglobulin polypeptide being selected to produce inhibition of breast cancer cell growth, and then contacting the cell with the anti-estrogen receptor immunoglobulin polypeptide under conditions which allow the anti-estrogen receptor immunoglobulin polypeptide to bind to the estrogen receptor associated with the surface membrane of the breast cancer cell to a degree sufficient to inhibit the growth of the breast cancer cell. In preferred embodiments of this method, the anti-estrogen receptor immunoglobulin polypeptide is selected from the group consisting of monoclonal antibodies Ab 1 and Ab 2.

In a variation of the methods utilizing anti-estrogen receptor immunoglobulin polypeptides discussed above, one can further contact the cell with an anti-HER-2 immunoglobulin polypeptide under conditions which allow the anti-HER-2 immunoglobulin polypeptide to bind to HER-2 on the surfaces of the breast cancer cell to a degree sufficient to inhibit the growth of the breast cancer cell. In yet another variation of the methods utilizing anti-estrogen receptor immunoglobulin polypeptides discussed above, one can further treat the cancer cell with anti-HER-1/EGF receptor immunoglobulin polypeptide or with a conventional therapy selected from the group consisting of surgical excision and chemotherapy.

Another embodiment of the invention disclosed herein includes an injectable pharmaceutical composition for treatment of a mammalian cancer tumor having cells which express estrogen receptor in association with their cell membranes consisting of an anti-estrogen receptor immunoglobulin polypeptide specific to an epitope on a ligand binding domain of the estrogen receptor; the anti-estrogen receptor immunoglobulin polypeptide being selected for its ability to inhibit tumor growth; and a pharmaceutically acceptable injection vehicle. Preferably, the anti-estrogen receptor immunoglobulin polypeptide is selected from the group consisting of monoclonal antibodies Ab 1 and Ab 2.

Yet another embodiment of the invention disclosed herein includes a kit for use in methods for inhibiting the growth of breast tumor cells which express an estrogen receptor comprising a container, a composition contained within the container, wherein the composition includes an anti-estrogen receptor immunoglobulin polypeptide and instructions for using the anti-estrogen receptor immunoglobulin polypeptide in vivo or in vitro. Preferably, the anti-estrogen receptor immunoglobulin polypeptide is selected from the group consisting of monoclonal antibodies Ab 1 and Ab 2.

Yet another embodiment of the invention disclosed herein includes a method of radioimaging metastasized breast cancer cells comprising the steps of first administering to an individual suspected of having metastasized breast cancer cells, a pharmaceutical composition that consists of a pharmaceutically acceptable carrier or diluent, and conjugated compound that comprises an anti-estrogen receptor immunoglobulin polypeptide and a radioactive active moiety wherein the conjugated compound is present in an amount effective for diagnostic use in humans suffering from breast cancer and then detecting the localization and accumulation of radioactivity in the individual's body.

### DESCRIPTION OF THE DRAWINGS

FIG. 1 is a schematic showing the postulated cellular mechanism of action of estrogen (E2) and growth factors in breast cancers with estrogen receptor (ER). In the classical model of estrogen action, estrogen binding to ER in the nucleus promotes receptor dimer formation and receptor phosphorylation that may enhance binding to nuclear estrogen-responsive elements (ERE) and coactivator proteins, leading, in turn, to initiation of specific gene transcription. However, the latter model fails to account for numerous, rapid cellular responses to estrogen treatment (see TABLE 1). In addition, although estrogen stimulates growth of breast cells, stable transfection of cells with the classical ER elicits a paradoxical estrogen-induced inhibition of growth (69, 70). These deficiencies in the classical model of estrogen action led to development of the current research proposal. In the hypothesis to be tested here, estrogen may also bind to a membrane-associated ER, with potential for stimulation of estrogenic responses via an alternate pathway. As noted in the text, membrane estrogen receptors may be known molecules (kinases, ion channels) with previously unknown binding sites for estrogen, new isoforms of ER-α or ER-β in membranes, classical ER complexed with other membrane-associated proteins or truly novel membrane-associated proteins.

FIG. 2 shows the distribution and relative specific activities of enzymes and specific [³H]estradiol-17β binding in plasma membrane and other subfractions of MCF-7 breast cancer cells. Cells were grown in estrogen-free media prior to harvesting, then disrupted using controlled homogenization methods as before (48,49). A) The yield of marker enzymes and E2β binding in each fraction is expressed as a percentage of that in the cell homogenate, with mean ±SE of data from 3 experiments shown. Total recoveries of protein, 5′-nucleotidase (5′-NUC), lactate dehydrogenase (LDH) and specific [³H]E₂β binding (E₂β) in crude nuclear (N), mitochondria-lysosome (ML), microsome-rich (Ms) and cytosol (S) fractions ranged from 96-102% of that in homogenates. Homogenate values averaged 34±2 mg/10⁸ cells for protein; 49±2 nmol/min/mg protein for 5′-nucleotidase; 48±4 units/min/mg protein for LDH; and 240±5 fmol/mg protein for specific[³H]-E₂β binding. B) Relative specific activity in plasma membrane (PM) represents the specific activity of enzyme or E₂β binding in a given fraction in relation to that in homogenates.

FIG. 3 is a photograph showing that estradiol-17β conjugated to fluorescein-labeled albumin (E₂β-BSA-FITC) binds at the surface membrane of MCF-7 breast cancer cells. After incubation with dextran-coated charcoal (DCC) to remove any traces of free estradiol, E₂β-BSA-FITC (1 μM) was used for cell labeling for 10 min and then analyzed by fluorescent microscopy. Control binding with inactive control ligand, BSA-FITC, shows a low level of background cell fluorescence of MCF-7 PAR cells (CONTROL). Active ligand, E₂β-BSA-FITC, labels surface membranes of MCF-7 PAR cells (PARENT:E2). As compared with parental cells, surface labeling appears reduced in MCF-7 HER-2 cells when incubated with active ligand, E₂β-BSA-FITC (HER-2:E2). In independent experiments, MCF-7 PAR and MCF-7 HER-2 cells were permeabilized with 0.1% Triton X-100 to allow visualization of ER binding in the nucleus (NUCLEAR). E2β-BSA-FITC is a steroid conjugate considered to be membrane-impermeant (52,60,61,94).

FIG. 4 is a graph showing that MCF-7 breast cancer cells proliferate in response to short-term treatment with membrane-impermeable estradiol-17β-bovine serum albumin conjugate (E2-BSA) or with free estradiol, but not with control BSA alone (P<0.001, t-test). Dextran-coated, charcoal-treated E2-BSA, a macromolecular complex used previously for affinity-isolation of estrogen receptor, is considered to be membrane-impermeant on brief exposure to cells (52,60,61, 94). Moreover, the proliferative effect of E2-BSA is blocked by treatment of cells with the pure antiestrogen, ICI 182,780.

FIG. 5 is a graph showing that estrogen elicits preferential growth of human breast cancer cells selected for expression of membrane-associated estrogen receptor. To assess the importance of membrane-associated ER in estrogen-induced cell growth, breast cancer cells were fractionated in vitro on the basis of their capacity to bind or not bind with 17β-estradiol-17-hemisuccinyl-albumin covalently bound to an inert support (46,47). Isolated MCF-7 breast cancer cells were cultivated in estrogen-free media for 72 hrs and then incubated for 30 min at 22 C. with immobilized estradiol at a prevailing concentration of approximately 0.5 nM as described before (47). These conditions were shown before to permit selection of cells with high affinity interactions with estradiol at the surface membrane (46,47). No significant binding of cells was observed when inert supports coupled only to albumin were used, indicating specificity of cell binding to immobilized estradiol. Breast cancer cells bound to immobilized estrogen (E2-binding) were dislodged from the fibers in the presence of excess E2β and recovered intact by centrifugation (47). Corresponding cells which had not become bound to immobilized estradiol (non-binding), as well as cells not selected for binding to immobilized estrogen (unfractionated) were processed and recovered under parallel conditions. All cell groups were than cultivated for 3d in estrogen-free media, followed by treatment with or without 2 nM estradiol-17β for 72 hrs. Cell numbers in all groups were quantitated and expressed relative to the initial cell number at the start of the treatments. The increment in estrogen-induced cell growth in E2-binding cells was significantly greater than that found in unfractionated and non-binding cell populations (P<0.001, t-test, 3 experiments).

FIG. 6 is a Western blot showing that the activation of HER-2 growth factor receptor promotes physical association of HER-2 receptor with estrogen receptor (ER). MCF-7 breast cancer cells were treated in vitro for 5-60 minutes with 10 nM heregulin, a ligand known to activate HER-2/HER-3 receptors (3). Lysates were prepared and processed as described before (3). Samples were immunoprecipitated with anti-HER-2 antibody (IP:HER-2 receptor) prior to electrophoresis and Western blotting with anti-ER antibody H222 (IB: estrogen receptor). Estrogen receptor normally occurs as a 65- to 70-kd protein (3). The experiment shown here is representative of results from 4 other experiments. In independent experiments in which samples were immunoprecipitated with anti-ER antibody prior to electrophoresis and Western blotting with anti-HER-2 antibody, a similar association between ER and HER-2 receptors was found.

FIG. 7 shows that treatment of human breast cancer cells with antisense oligonucleotides targeting nuclear ER mRNA reduce the expression of total cellular ER. The groups include: CON (control), AS (antisense phosphorothioate 5′ GGGTCATGGTCATGG; SEQ ID NO: 1) and Ms (missense control).

FIG. 8 is a graph showing that treatment of human breast cancer cells with antisense oligonucleotides to intracellular estrogen receptor suppress expression of membrane-associated receptors with specific high-affinity binding for estrogen. The antisense phosphorothioate oligonucleotide was synthesized as 5′-GGGTCATGGTCATGG-3′ (SEQ ID NO: 1), and a missense control was used for comparison. Specific estradiol-17β binding to plasma membrane fractions was done by established methods.

FIG. 9 is a graph showing that treatment of human breast cancer cells with ER antisense oligonucleotides reduce the expected cellular growth response to 2 nM estradiol-17β. Treatment groups include: control cells (Cn), control cells treated with estradiol (Cn/E), missense oligonucleotide treated cells (Ms), Ms-treated cells with exposure to estradiol (Ms/E), ER antisense-treated cells (As), and As-treated cells exposed to estradiol (As/E).

FIG. 10 is a schematic showing potential sites for estrogen action in the cell. Estrogen may interact with a membrane-associated estrogen receptor (ER), leading to signal transduction to the cell interior by interactions with receptor tyrosine kinases (RTK), MAP kinase cascades or G-protein related pathways. Interference with estrogen-ER interactions or with the activity of downstream signaling pathways may block estrogen-stimulated effects in the cell.

FIG. 11 is a graph showing that the MAPKK (MEK) inhibitor, PD 98059, reduces estrogen-stimulated MAPK activity. MAPK activity was assessed in human breast cancer cells after treatment with estradiol (E2) alone or combined with 20 μM PD 98059 (E2/PD) over 30 min. Results are presented as percent of appropriate controls.

FIG. 12 is a gel showing that the MAPKK (MEK) inhibitor, PD 98059 (PD), reduces estrogen (E2)-induced serine phosphorylation of estrogen receptor. Serine phosphorylation of estrogen receptor in human breast cancer cells was assessed at 15-60 min after treatment with E2 alone or in combination with PD 98059

FIG. 13 is a graph showing that MAPKK (MEK) inhibitor PD 98059 (PD) reduces estrogen (E2)-induced growth of breast cancer cells. MCF-7 human breast cancer cells were treated in vitro with 20 micromolar PD 98059, a synthetic inhibitor of the MAPK-activating enzyme, MAPK/ERK kinase (MEK) (Proc. Natl. Acad. Sci. USA 92: 7686-7689, 1995) in the presence and absence of estradiol-17β at 2 nM. Control (CN); estradiol (E2); PD 98059 alone (CN/PD); estradiol and PD 98059 (E2/PD).

FIG. 14A is table which provides information on properties and sources of the various antibodies used in the methods of the invention.

FIG. 14B is a schematic of the estrogen receptor-alpha protein structure which outlines the various identified functional domains within this molecule.

FIG. 15 shows binding of [³H]estradiol-17β by plasma membranes from MCF-7 human breast cancer cells. A) Plasma membranes were incubated in Ca⁺⁺-free medium with 0.25 M sucrose with proteinase inhibitors at 50 μg membrane protein/2.5 ml for 2 h at 4° C. with the concentrations of [³H]E₂β given alone (curve x) or in the presence of a 100-fold molar excess of unlabeled E₂β plus [³H]E₂β (curve y). B) This curve shows the difference between the two curves in panel a and represents the specific binding of hormone by plasma membranes. In the inset, ligand specificity of [³H]estradiol-17β binding was determined by incubation in the presence of a 100-fold molar excess of competing steroidal compounds: E₂β, E₂α, progesterone (PRG) or testosterone (TST) as indicated in the graph. Values are shown as mean percent control ±SE(n=3).

FIG. 16 shows the identification of estrogen receptor in subcellular fractions of MCF-7 cells by Western blot and ligand-blot analyses. Proteins from cell subfractions were analyzed by polyacrylamide gel electrophoresis and transferred to nitrocelulose membranes. A) Immunoblotting with a monoclonal antibody against the LBD of nuclear ER shows the presence of a major 67-kDa band in the homogenate (H) as well as in nuclear (N), mitochondria-lysosome (ML), microsome (Ms) and cytosol (S) fractions. Notably, a band of similar molecular size also shows enrichment in plasma membrane fractions (PM). B) Using a ligand-blot approach, binding of E₂β-POD to a 67-kDa band is likewise found to be enriched in plasma membranes (PM) and in nuclear (N) fractions. E₂β-POD binding is shown in the absence (none) and presence (E₂β) of free estradiol-17β at a 10-fold molar excess in order to assess specific steroid binding (103).

FIG. 17 is a gel showing that estrogen treatment promotes enhanced association between ER-α and Cavatellin-2/Flotillin-2/ESA in MCF-7 human breast cancer cells. Breast cancer cells were treated with vehicle alone (VH) or 10 nM 17β-estradiol (E₂β) for 1, 5, 10 and 20 min. Thereafter, cells were lysed and immunoprecipitated (IP) with anti-estrogen receptor-alpha (ER-α) antibody. Immunoprecipitates were subjected to Western blot (IB) with anti-Cavatellin-2/Flotillin-2/ESA antibody

FIG. 18 shows post-receptor signal transduction induced by estradiol in vitro. A) Treatment of MCF-7 cells with 10 nM estradiol-17β (E₂β) induces rapid phosphorylation of mitogen-activated protein kinase (MAPK). E₂β, but not 17α-estradiol (E₂α) or vehicle control (CN), promotes phosphorylation of MAPK isoforms, extracellular signal-regulated kinase ERK-1 (p44) and ERK-2 (p42), with effects evident within 2 min. Similarly, MCF-7 cells treated with E₂β covalently linked to BSA (E₂β-BSA, 0.5 μM), but not to control E₂α-BSA (0.5 μM), promoted MAPK phosphorylation within 2 min. Prior treatment with antibody to the LBD of ER (Ab2) blocked the expected response to E₂β (Ab2+E₂β) and to E₂β-BSA (Ab2+E₂β-BSA). In addition, cells were preincubated with U0126, a selective inhibitor of MEK1 and MEK2, before treatment with estrogens, and the inhibitor prevented activation of MAPK by E₂β (UO126+E₂β) and by E₂β-BSA (UO126+E₂β-BSA). B) Akt kinase activation was measured by densitometric analysis of phosphorylated GSK-3α/β. MCF-7 cells were treated with vehicle (CN) or stimulated with 10 nM estrogen (E₂β) or 0.5 μM E₂β-BSA for 20 min. Cells were preincubated with anti LBD Ab2 (Ab2), ER antagonist ICI 182,780 (ICI) or the PI3-kinase inhibitor LY294002 (LY) before addition of E₂β-BSA.

FIG. 19 shows that estradiol-17β conjugated to fluorescein-labeled albumin (E₂β-BSA-FITC) shows binding at the surface membrane of MCF-7 breast cancer cells. E₂β-BSA-FITC was pre-treated with dextran-coated charcoal to remove any traces of free estradiol. Thereafter, intact cells were labeled with 1 μM E₂β-BSA-FITC, a membrane-impermeant complex (52,60,61,94), to assess membrane binding and then analyzed by fluorescent microscopy and flow cytometry. a) Active ligand, E₂β-BSA-FITC, labels surface membranes of MCF-7 cells. b) Control binding with inactive ligand, BSA-FITC, shows a low level of background cell fluorescense. c) Surface membrane labeling by E₂β-BSA-FITC is competitively reduced by co-incubation with antibody to LBD of ER (Ab1). d) MCF-7 cells were permeabilized with 0.1% Triton X-100 to allow visualization of ER binding in the nucleus. e) Flow cytometric analysis of membrane fluorescense with E₂β-BSA-FITC. Cells were incubated with BSA-FITC for background fluorescense. With 10,000 cells analyzed per sample, a significant decrease (P<0.01) in fluorescense intensity was observed when cells were incubated with estrogen (E₂β), E₂β-BSA, tamoxifen (TAM), ICI 182,780 (ICI) or anti ER antibody (Ab1). No significant competition was observed when cells were incubated in the presence of progesterone (PRG). In additional control studies, COS-7 monkey kidney cells with no ER showed no significant binding or retention of E₂β-BSA-FITC label.

FIG. 20 shows the inhibition of MCF-7 breast cancer cell growth by a monoclonal antibody directed against the LBD of nuclear ER. A) Cells were incubated in vitro for 2 h with anti-ER antibodies directed against the LBD (Ab1 and Ab2) or with a control antibody directed to the D and E-domains of ER (Ab3). Thereafter, E₂β, 17α-estradiol (E₂α), E₂β-BSA or E2α-BSA were added to cultures for 10 min. Cells were then cultivated further, and final cell numbers were quantitated after 72 h for each treatment group as indicated. Data (mean±SE) were collected from at least 4 independent experiments. B) Monoclonal antibody directed against the LBD of ER-α reduces growth of human MCF-7 breast tumor cell xenografts in vivo. Female nude mice were primed by treatment with E₂β subcutaneously, then inoculated with MCF-7 cells as before (3). After 10-14 days, animals with tumors of comparable size were randomized to treatment groups of 6-8 mice. Treatments included IgG isotype-control antibody (CON) or monoclonal antibody directed against the LBD of ER-α (Ab2) administered intraperitoneally twice weekly for a total of 6 doses. After 26 days, no further antibody treatment was given. Tumor volumes were recorded by micrometer measurements, with results shown as mean±SE.

FIG. 21 is a graph showing Inhibition of MCF-7 human breast cancer cell growth by the MEK 1/2 inhibitor U0126. Cells were incubated in vitro in the presence of 10 μM U0126 (Favata, M F et al., 1998, J. Biol. Chem. 273, 18623-18632) for 2 h before treatment with vehicle alone (VH), 10 nM estradiol 17-β (E₂) or 1 μM of E₂ coupled to serum bovine albumin (E₂-BSA) for 10 min. Cells were then cultivated further, and final cell numbers were quantitated after 72 h.

### DETAILED DESCRIPTION OF THE INVENTION

Membrane-associated forms of estrogen receptor play an important role in promoting intracellular signaling for hormone-mediated proliferation and survival of breast cancers and offer a new target for antitumor therapy. As disclosed herein, estrogen-induced growth of cancer cells is blocked by treatment with antibodies directed to the ligand-binding domain of the nuclear form of estrogen receptor. Moreover, this effect correlates closely with acute estrogen-induced activation of mitogen-activated protein kinase (MAPK) and Akt kinase signaling, and estrogen-induced growth of cancer cells is also blocked by synthetic inhibitors of MAP kinase-activating enzymes. Significantly, estrogen-promoted growth of human breast cancer xenografts in nude mice was significantly reduced by treatment in vivo with antibodies directed to the ligand-binding domain of the estrogen receptor. Thus, antibody directed to membrane-associated estrogen receptor may be one strategy for disrupting cell growth. However, other therapeutic strategies may include the use of agents that can act as partial agonists or antagonists of the membrane receptor (for example, ICI 182,780) or drugs that block estrogen-induced activation of MAPK or PI3K/Akt kinase signaling cascades that emanate from stimulation of the membrane receptor. Specific evidence for the efficacy of these alternate approaches is presented in the Examples below.

The present invention is directed to methods of inhibiting the signalling of a plasma membrane-associated estrogen receptor by contacting the membrane-associated estrogen receptor with an inhibitory ligand that binds to the ligand-binding domain of the membrane-associated estrogen receptor thereby inhibiting plasma membrane-associated estrogen receptor signalling. The invention provides a new approach to the treatment of cancers that express membrane-associated estrogen receptors, for example breast cancers. In particular, we disclose that the treatment of human breast cancers with antibodies directed to the ligand binding domain of the human estrogen receptor can block the growth of breast cancer cells that bear estrogen receptors. Similarly, treatments targeted to downstream signaling events after stimulation of the membrane estrogen receptor also appear to suppress tumor growth. As approximately 75% of human breast cancers express the estrogen receptor at the time of cancer diagnosis, this approach to treatment with blood-borne reagents potentially represents an important addition to available treatment options.

The invention disclosed herein challenges the current dogma in endocrinology and oncology which holds that estrogen receptors are exclusively intracellular proteins and largely localized to the nucleus of the cell. Specifically, we demonstrate that the use of anti-estrogen receptor immunoglobulin polypeptides targeting the membrane-associated estrogen receptor expressed by these cells elicits a potent antitumor effect. These observations of membrane-associated estrogen receptors on human breast tumor cells may be equally relevant to other estrogen-regulated processes in diverse tissues such as bone, brain, uterus and vasculature.

The invention disclosed herein has advantages over existing treatment practices: Patients with breast cancer are usually offered multimodality therapy, including surgery, radiation and adjuvant treatment. Depending on tumor stage and estrogen receptor status of the tumor at the time of presentation, adjuvant therapy may include antiestrogen treatment (tamoxifen) or cytotoxic chemotherapy. All current adjuvant treatments are encumbered by numerous side-effects that often reduce the patient's quality of life. Membrane-associated estrogen receptor targeted therapy could provide a new alternative with fewer side-effects and improved quality of life for the affected patients.

Optimal embodiments of the methods of the inventions entail those with blockade of estrogen receptor function and tumor cell growth by use of circulating monoclonal antibodies to the estrogen receptor may offer the best outcome. This form of therapy could prove to be analogous to the use of Herceptin, a monoclonal antibody directed to the surface membrane receptor termed HER-2/neu. See e.g. U.S. Pat. Nos. 5,772,997, 5,824,311 and 5,773,476 which are incorporated herein by reference. In addition, blockade of intracellular signaling emanating from activation of the membrane-associated estrogen receptor would offer another method of the invention for inhibition of tumor cell growth.

I. Definitions

The terms “peptide”, “polypeptide” or “protein” are used interchangeably herein. The term “substantial identity”, when referring to polypeptides, indicates that the polypeptide or protein in question is at least about 30% identical to an entire naturally occurring protein or a portion thereof, usually at least about 70% identical, and preferably at least about 95% identical.

As used herein, the terms “isolated”, “substantially pure” and “substantially homogenous” are used interchangeably and describe a protein that has been separated from components which naturally accompany it. Typically, a monomeric protein is substantially pure when at least about 60 to 75% of a sample exhibits a single polypeptide backbone. Minor variants or chemical modifications typically share the same polypeptide sequence. A substantially purified protein will typically comprise over about 85 to 90% of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band on a polyacrylamide gel upon staining. For certain purposes high resolution will be needed and HPLC or a similar means for purification utilized.

A polypeptide is substantially free of naturally-associated components when it is separated from the native contaminants which accompany it in its natural state. Thus, a polypeptide which is chemically synthesized or synthesized in a cellular system different from the cell from which it naturally originates will be substantially free from its naturally-associated components. Proteins may be purified to substantial homogeneity by standard techniques well known in the art, including selective precipitation with such substances as ammonium sulfate, column chromatography, immunopurification methods, and others. See, for instance, R. Scopes, Protein Purification: Principles and Practice, Springer-Verlag: N.Y. (1982), which is incorporated herein by reference.

As used herein, “immunoglobulin polypeptide” refers to molecules which have specific immunoreactive activity. Antibodies are typical embodiments of immunoglobulin polypeptides.

As used herein, the term “antibody” refers to a protein consisting of one or more polypeptides substantially encoded by immunoglobulin genes. Immunoglobulin genes include those coding for the light chains, which may be of the kappa or lambda types, and those coding for the heavy chains. Heavy chain types are alpha, gamma, delta, epsilon and mu. The carboxy terminal portions of immunoglobulin heavy and light chains are constant regions, while the amino terminal portions are encoded by the myriad immunoglobulin variable region genes. The variable regions of an immunoglobulin are the portions that provide antigen recognition specificity. In particular, the specificity resides in the complementarity determining regions (CDRs), also known as hypervariable regions, of the immunoglobulins. The immunoglobulins may exist in a variety of forms including, for example, Fv, Fab, F(ab′), F(ab′)₂, and other fragments, as well as single chains (e.g., Huston, et al., Proc. Nat. Acad. Sci. U.S.A., 85:5879-5883 (1988) and Bird, et al., Science 242:423-426 (1988), which are incorporated herein by reference). (See, generally, Hood, et al., “Immunology”, Benjamin, N.Y., 2nd ed. (1984), and Hunkapiller and Hood, Nature, 323:15-16 (1986), which are incorporated herein by reference).

“Monoclonal antibodies” are well known in the art and may be obtained by various techniques familiar to those skilled in the art. A monoclonal antibody is an antibody produced by a clonal, immortalized cell line separate from cells producing antibodies with a different antigen binding specificity. Briefly, spleen cells from an animal immunized with a desired antigen are immortalized, commonly by fusion with a myeloma cell (see, Kohler and Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Monospecific and bispecific immunoglobulins may also be produced by recombinant techniques in prokaryotic or eukaryotic host cells.

“Chimeric” antibodies are those antibodies encoded by immunoglobulin genes that have been genetically engineered so that the light and heavy chain genes are composed of immunoglobulin gene segments belonging to different species. For example, the variable (V) segments of the genes from a mouse monoclonal antibody may be joined to human constant (C) segments. Such a chimeric antibody is likely to be less antigenic to a human than antibodies with mouse constant regions as well as mouse variable regions. As used herein, the term “chimeric antibody” also refers to an antibody that includes an immunoglobulin that has a human-like framework and in which any constant region present has at least about 85-90%, and preferably about 95% polypeptide sequence identity to a human immunoglobulin constant region, a so-called “humanized” immunoglobulin (see, for example, PCT Publication WO 90/07861, which is incorporated herein by reference). Hence, all parts of such a “humanized” immunoglobulin, except possibly the complementarity determining regions (CDR's), are substantially identical to corresponding parts of one or more native human immunoglobulin sequences. Where necessary, framework residues may also be replaced with those within or across species especially if certain framework residues are found to affect the structure of the CDRs. A chimeric antibody may also contain truncated variable or constant regions.

The term “framework region”, as used herein, refers to those portions of immunoglobulin light and heavy chain variable regions that are relatively conserved (i.e., other than the CDR's) among different immunoglobulins in a single species, as defined by Kabat, et al., (1987): Sequences of Proteins of Immunologic Interest, 4th Ed., US Dept. Health and Human Services, which is incorporated herein by reference). As used herein, a “human-like framework region” is a framework region that in each existing chain comprises at least about 70 or more amino acid residues, typically 75 to 85 or more residues, identical to those in a human immunoglobulin.

II. Modulation of Plasma Membrane-Associated Estrogen Receptor Signalling

Growth factor receptor malfunction occurs in malignant progression, with members of the HER-1 (EGF) family most frequently implicated in human cancer (1-3, 4-8). The HER (erb B) receptor family includes the HER-2 (erb B2) protein, a 185-kD transmembrane tyrosine kinase encoded by HER-2 oncogene, (9-11), the HER-3 protein (12) and HER-4 receptor (13,14). Overexpression of HER-2 or related growth factor receptors is estimated to occur in two-thirds of sporadic breast cancers (1), while amplification or overexpression of HER-2 is found in 25-30% of breast cancers in women and 41% of breast cancers in men (15-18). HER-2 overexpression is a marker of poor prognosis (15-19) and is associated with the failure of antiestrogen therapy (3,20-31).

Receptors for estrogen also occur in a family of potentially oncogenic receptors. Sequence similarities between the erb A gene product of avian erythroblastosis virus and ER provide evidence that these two proteins likely evolved from a common gene (32). Erb A genes cannot induce cell transformation alone, but cooperate with the viral erb B oncogenes in cell transformation (33). With this lineage of cooperativity between erb A and erb B genes, it is not surprising to find reports of significant cross-communication and interaction between erb B (HER) pathways and estrogen receptor signaling (3,24,27,34-36).

It is generally held that the biologic activity of estrogen in the breast is mediated through the specific high-affinity estrogen receptor located in breast cell nuclei (1,37). In the absence of estrogen, ER is considered to associate with proteins that prevent its interaction with the cellular transcription apparatus. Upon estrogen binding, the receptor undergoes an activating conformational change that promotes association with target genes, thus permitting regulation of gene transcription.

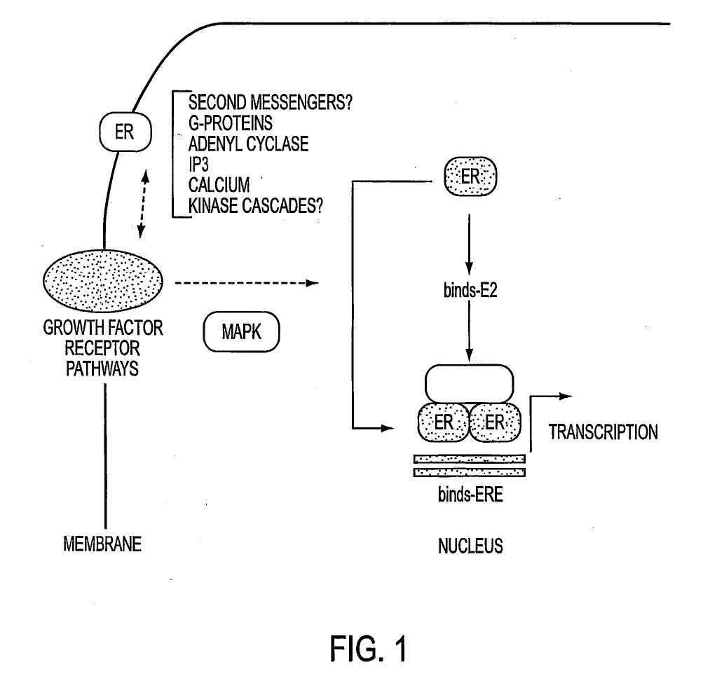

FIG. 1 shows a schematic of the postulated cellular mechanism of action of estrogen (E2) and growth factors in breast cancers with estrogen receptor (ER). In the classical model of estrogen action, estrogen binding to ER in the nucleus promotes receptor dimer formation and receptor phosphorylation that may enhance binding to nuclear estrogen-responsive elements (ERE) and coactivator proteins, leading, in turn, to initiation of specific gene transcription. However, the latter model fails to account for numerous, rapid cellular responses to estrogen treatment (see TABLE 1).

In addition, although estrogen stimulates growth of breast cells, stable transfection of cells with the classical ER elicits a paradoxical estrogen-induced inhibition of growth (69,70). These deficiencies in the classical model of estrogen action led to development of the disclosure herein that estrogen also binds to a plasma membrane-associated ER, with potential for stimulation of estrogenic responses via an alternate pathway. Membrane-associated estrogen receptors may be known molecules (kinases, ion channels) with previously unknown binding sites for estrogen, new isoforms of ERα or ERβ in membranes, classical forms of ER complexed with other membrane-associated proteins, truly novel membrane proteins, or a combination of these options.

Current reports provide evidence that membrane ER may activate one or more of several pathways, including interaction with growth factor membrane receptors such as HER-2 tyrosine kinase receptor or activation of G-proteins and adenylate cyclase, inositol phosphate, calcium homeostasis and/or MAP kinase (see TABLE 1 for details). These membrane interactions promote phosphorylation of ER via estrogen-induced activation of second-messengers and protein kinases or, alternatively, via ligand-independent pathways involving growth factor receptors. Growth of cells treated with estrogen may occur as a consequence of a synergistic feed-forward circuit where estrogen activates cell membrane signaling pathways that act, in turn, to enhance the transcriptional activity of ER in the nucleus (see TABLE 1). Active reconsideration of the classical model of nuclear receptor action is ongoing (38), and the importance of alternate signaling pathways is only now beginning to emerge. The outcome of this academic debate has important ramifications for antiestrogen therapy of human breast cancer in the clinic.

TABLE 1
Summary of selected reports for existence and activity
of a plasma membrane-associated estrogen receptor.

| Year | Observation | Reference |
|---|---|---|
| 1967 | Acute stimulation of cAMP by estrogen | 41 |
| 1977 | Specific plasma membrane binding sites for estrogen | 46 |
| 1979 | Rapid calcium mobilization by estrogen | 39 |
| | Membrane estrogen receptors regulate proliferation | 47 |
| 1980 | Estrogen receptors in liver plasma membrane | 48 |
| | Specific estrogen binding to breast cancer cell | 49 |
| | membrane | |
| 1981 | Cell surface immunologic blockade of estrogen action | 50 |
| 1984 | Estrogen receptors in endometrial plasma membrane | 51 |
| 1986 | Plasma membrane binding sites for estrogen in | 52 |
| | breast cancer | |
| 1993 | Cell signaling and non-nuclear estrogen receptors | 53 |
| | Estrogen stimulates autophosphorylation of HER-2 | 54 |
| | receptor | |
| 1994 | Estrogen action via the cAMP signaling pathway | 42 |
| 1995 | Membrane estrogen receptors and rapid membrane | 55 |
| | responses | |
| | Membrane estrogen receptors by antibody labeling | 56 |
| | Nongenomic effects of estradiol-17β | 57 |
| 1996 | Membrane estrogen binding and nongenomic effects | 58 |
| 1997 | Membrane action of estradiol | 45 |
| | Rapid membrane effects of estrogen and MAP kinase | 59 |
| | signaling | |
| | Membrane estrogen-binding proteins with different | 60 |
| | molecular sizes by ligand blotting method | |
| 1999 | Nongenomic activity of estrogen on calcium and | 40 |
| | MAP kinase | |
| | Membrane and nuclear ERα and ERβ from single | 61 |
| | transcript | |
| | Antisense oligonucleotides to nuclear ER block | 92 |
| | expression of membrane ER and disrupt breast tumor | |
| | growth | |

In addition to the pathways typically discussed in the art, it has been established that estrogen can also induce extremely rapid increases in the levels of intracellular second messengers, including calcium (39,40) and cAMP (41,42), as well as activation of mitogen-activated protein kinase (43,44) and phospholipase (45) [see TABLE 1]. The time course of these events is similar to those elicited by peptides, lending support to the hypothesis that they do not involve the classical genomic action of estrogen. Both estrogens and growth factor ligands act as mitogens to promote cell growth in the breast, and the cellular effects of these agents sometimes overlap. The molecular details of this cross-talk between ER and erb B receptors are now beginning to emerge, and ER itself may be an important point of convergence (3,24,34-36).

Many of the rapid effects of estrogen appear to be due to the action of the hormone at the cell membrane, and these biologic actions appear to be mediated by plasma membrane-associated receptors that bind estrogen. The isolation and complete structural characterization of these native macromolecules have not yet been accomplished, and the derivation and functions of this receptor (or receptors) remain to be fully established. Since activation of this alternate signaling pathway by estrogens may represent a mechanism by which estrogens regulate proliferation, we have investigated the nature and activity of this membrane response pathway in human breast cancer cells. Classical models of estrogen action that characterize this signaling pathway as solely due to the activity of an intracellular ligand-dependent transcription factor are clearly incomplete and must be modified to include estrogen receptors as significant components of other signaling pathways.

Antiestrogen therapy is commonly used for treatment of patients with breast cancers that express estrogen receptor (ER). The efficacy of endocrine treatment depends on the close regulation of breast cell growth by estrogens and peptide growth factors. However, as breast cancer progresses, the disease usually becomes resistant to estrogens, and most patients no longer respond to antiestrogen therapy. New options for antiestrogen treatment are clearly needed, and alternative therapies may now derive from findings showing that a portion of ER molecules occur in plasma membranes of human breast cancer cells and may interact with transmembrane erb B/HER growth factor receptors. It is known that expression of HER-2/erb B-2 receptors occurs in many human breast cancers, and the protein kinase activity of HER-2 may play a role in the ligand-independent activation of ER. If active cross-communication between ER and HER-2 growth factor receptor occurs and leads to promotion of cancer growth, this signaling complex may offer an important new target for therapeutic intervention. Since overexpression of HER-2 receptor in breast cancer is associated with the failure of antiestrogen therapy in the clinic, understanding the biologic basis of the association between membrane estrogen receptors and HER-2 receptors may also help to improve decisions on patient management and to increase patient survival. As illustrated below, the disclosure herein both provides insight into the biology of breast cancer as well as providing novel methods for inhibiting the growth of breast cancer cells.

In the clinic, endocrine therapy is an important intervention in women with breast cancers that express the estrogen receptor (ER). Treatment with tamoxifen and other antiestrogens has increased the survival of breast cancer patients, and these agents are now used in breast cancer prevention. The success of endocrine therapy in breast cancer is dependent on the close regulation of breast cell growth by steroid hormone and growth factor receptors (1,2). However, as breast cancer progresses, it usually becomes resistant to estrogens, and, consequently, most patients no longer respond to therapy with tamoxifen or other antiestrogens. As disclosed herein, new information on the existence of an alternate estrogen signaling pathway in breast cancer cells allows the design of novel and more effective antihormone treatments for human breast cancers (3).

As shown in the Examples below, the disclosure herein provides evidence for the existence of receptors for estrogen in association with plasma membranes of human breast cancer cells. By use of controlled subcellular fractionation, plasma membranes were isolated from human breast cancer cells with and without overexpression of HER-2 receptor. Activity of estrogen receptors was found by ligand binding and immunoassay with antibody to ER Using an independent approach, we find that antisense oligonucleotides directed to nuclear ER block expression of both nuclear and membrane forms of ER. This provides evidence that membrane ER may originate, in part, from the same transcripts that produce the intracellular ER. Purification of membrane-associated estrogen receptors may be achieved by affinity chromatography, with recovered receptor to be used for preparation of new monoclonal antibodies and for further molecular characterization.

As shown in the Examples below, the disclosure herein further provides evidence that membrane estrogen receptors play a role in promoting the growth of human breast cancers. These studies provide new evidence of signal transduction by membrane ER, including interactions with HER-2 trans-membrane receptor and other signaling pathways such as mitogen-activated protein kinase. Moreover, by suppression of membrane ER expression with antisense oligonucleotides, we find a blockade of the growth of breast cancer cells. Using this evidence, one can evaluate downstream effects on calcium homeostasis, G protein activation, adenylate cyclase activity, inositol phosphate production, activation of HER-2 kinase and the association of these events with the activation of transcription and growth in breast cancer cells.

As shown in the Examples below, the disclosure herein further provides evidence that treatments directed to membrane-associated estrogen receptors lead to the blockade of breast cancer cell growth. Specifically, cells with defined levels of ER-α, membrane ER and HER-2 receptors show significant growth inhibition in vitro after treatment with antibodies directed to the ligand binding domain of ER. Treatment of breast cancer cells with membrane-impermeant estradiol (estradiol linked to BSA) leads to the rapid stimulation of MAP kinase activity in the cell interior, an event that may be associated with the promotion of tumor cell growth. By targeting the inhibition of downstream MAP kinase in estrogen-stimulated cells, blockade of tumor growth occurs. Further understanding of how these membrane receptor pathways function in the cancer cell may aid patient management and treatment decisions in the clinic. In this context, one can assess the potential therapeutic application of antibodies directed to membrane-associated estrogen receptors, alone and in combination with antibodies to other molecules associated with oncogenic processes, for example the HER-2 or HER-1/EGF receptors. In challenging the conventional dogma of estrogen action exclusively via an intracellular receptor, this work may lead to development of previously unsuspected, less toxic antitumor therapies targeted to human breast cancer cells.

As disclosed herein, membrane-associated binding sites for estrogen may mediate rapid effects of estradiol-17β that contribute to proliferation of human breast cancers. After controlled homogenization and fractionation of MCF-7 breast cancer cells, the bulk of specific estradiol binding is found in nuclear fractions. However, a significant portion of specific, high-affinity estradiol-17β binding-sites are also enriched in plasma membranes. These estradiol binding-sites co-purify with 5′-nucleotidase, a plasma membrane-marker enzyme, and are free from major contamination by cytosol or nuclei. Electrophoresis of membrane fractions allowed detection of a primary 67-kDa protein and a secondary 46-kDa protein recognized by estradiol-17β and by a monoclonal antibody directed to the ligand-binding domain of the nuclear form of estrogen receptor. Estrogen-induced growth of MCF-7 breast cancer cells in vitro was blocked by treatment with the antibody to estrogen receptor and correlated closely with acute hormonal activation of mitogen-activated protein kinase and Akt kinase signaling. Estrogen-promoted growth of human breast cancer xenografts in nude mice was also significantly reduced by treatment in vivo with the estrogen receptor antibody. Thus, membrane-associated forms of estrogen receptor plays a role in promoting intracellular signaling for hormone-mediated proliferation and survival of breast cancers and offer a new target for antitumor therapy.

As disclosed herein, we demonstrate that human breast cancer cells contain a membrane-associated binding site for estrogen that closely resembles nuclear ER. Activation of this membrane-associated receptor appears to promote rapid stimulation of MAPK and PI3K/Akt kinase signaling and later cell proliferation. Biologic activity of the membrane-associated receptor can be diminished in vitro and in vivo by antibody directed against the ligand-binding domain of nuclear ER. The results provide evidence that estrogens may initiate membrane-associated signaling events leading to modulation of the growth and survival of breast cancers.

In isolating and purifying membrane-associated estrogen-binding proteins from breast cancer cells, this work further elucidates molecular properties of the membrane-associated receptor in malignant cells. After use of controlled cell fractionation procedures to preserve the integrity of subcellular structures (47,48) the bulk of specific E₂β binding in MCF-7 cells is found in nuclear fractions. However, a significant portion of specific E₂β-binding sites also occur in association with plasma membranes. These E₂β binding-sites co-purify with 5′-nucleotidase, a plasma membrane-marker enzyme, and appear to be free from significant contamination by cytosol or nuclei. The plasma membrane E₂β binding-sites constitute about 20% of total cell binding-sites for the steroid, a level of membrane concentration comparable to that found for other known transmembrane hormone receptors (92). In addition, monoclonal antibodies against the LBD of nuclear ER can identify membrane-associated ER in MCF-7 cells, a finding consistent with studies with other cell types (56,61,94). The primary membrane-associated protein reactive with antibodies to LBD of nuclear ER and with E₂β ligand is 67-kDa, a molecular size comparable to that of nuclear ER, but additional protein species, notably at 46-kDa, were also detected (32,93). Two forms of ER-α with molecular masses of 67- and about 46-kDa occur in target cells, including vascular endothelial cells (94) and MCF-7 cells (95), and in HeLa cells transfected with ER cDNA (32). The nature of the truncated receptor form may be due, in part, to limited protein degradation or to alternative translation (95). These data provide evidence that membrane-associated estrogen-binding proteins contain common structural elements, at least in key molecular domains, with ER-α and various splice variants.

The disclosure herein confirms membrane-initiated signal transduction by ER in breast cancer cells, including interactions with signaling pathways such as MAPK and PI3K/Akt kinase. Independent reports showing a lack of MAPK activation in the absence of ER (40, 61) and the ability of pure antiestrogen, ICI 182,780, to inhibit estrogen-induced MAPK activity in MCF-7 cells (40) strongly implicate ER in this pathway and define a potentially important link between estradiol and the cell cycle. These stimulatory effects of free E₂β appear to be equaled by E₂β-BSA in vitro. In addition, the serine/threonine kinase Akt, a downstream effector of PI3-kinase, has been implicated in cell survival and prevention of apoptosis in MCF-7 cells (96).

The activation of membrane-associated forms of ER contribute to promotion of tumorigenesis of breast cancers. Treatment of breast cancers with macromolecular antibodies directed to the LBD of nuclear ER block the growth of tumors that bear functional ER. This finding is supported by the report by Norfleet (97) showing that antibodies to ER can modulate rapid prolactin release from pituitary tumor cells with membrane-associated ER. Further study will be required to assess the efficacy of ER antibodies in larger established tumors. Nevertheless, these findings offer support for earlier reports showing that estrogen-induced membrane signaling leads to the later activation of DNA synthesis and cell growth (61). It is likely that primary E₂β-induced activation of membrane-associated ER will also affect subsequent hormonal interactions with nuclear ER to promote activation of transcription and cell proliferation. Similarly, the molecular details of cross-communication between estrogen and peptide receptors are beginning to emerge (51,99,3), and membrane ER appears to be in a pivotal cellular location to enhance convergence among diverse signaling pathways. Since more than 60% of human breast cancers express ER at diagnosis (98), biologically-based therapies in the form of antiestrogens have been a mainstay in breast cancer treatment A novel approach to antitumor therapy with blood-borne anti-receptor agents represents an important addition to available treatment options. In promoting a model of estrogen action via both nuclear and membrane-associated receptors, this work may lead to development of previously unsuspected antitumor therapies targeted to breast cancers.

The finding that breast cancer cells show significant growth inhibition in vitro and in vivo after treatment with inhibitory ligands such as antibodies directed to the ligand binding domain of membrane associated ER is surprising in view of the prevailing dogma that the active estrogen receptor is active in the nucleus of the cell. Moreover, as one cannot predict which portions of the membrane-associated estrogen receptor (previously thought to be active in the nucleus of the cell) would be exposed at the cell surface and contain a region capable of being recognized by a ligand such as antibody, the finding that such regions of membrane associated ER are exposed and that antibodies targeting that particular domain of the estrogen receptor have the greatest inhibitory effects is also unexpected. In addition, because the estrogen receptor is generally involved in the activation of cell growth, one could not predict the finding that molecules that interact with this receptor (particularly those that interact with the ligand binding domain) would inhibit receptor-mediated activity.

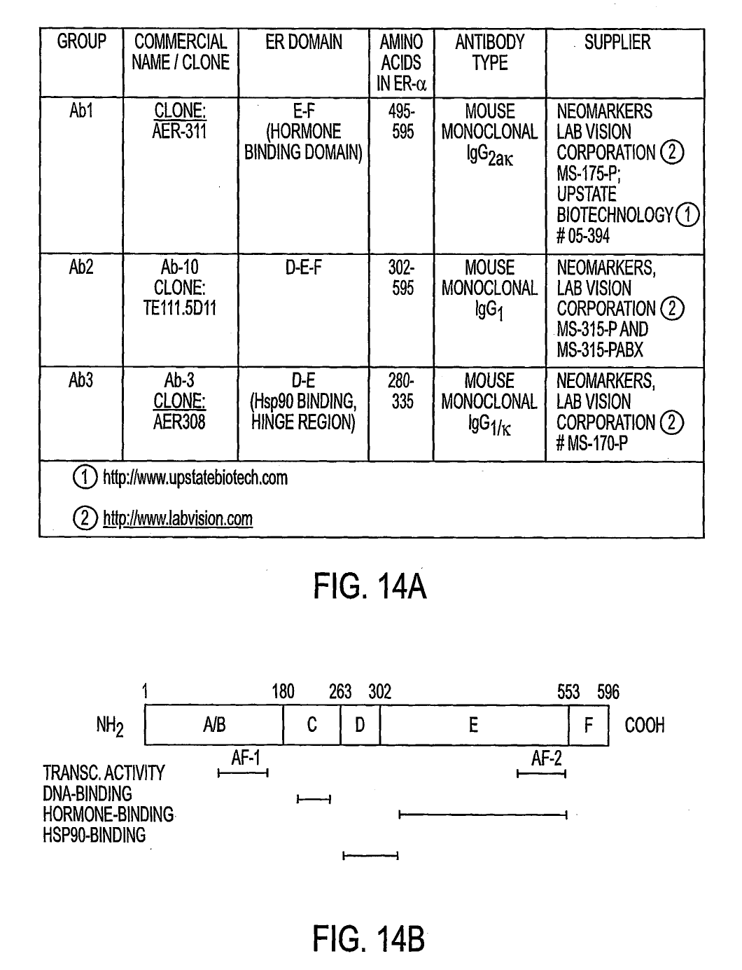

An illustrative embodiment of the invention provided herein comprises a method of inhibiting the signalling of a plasma membrane-associated estrogen receptor comprising contacting the membrane-associated estrogen receptor with an inhibitory ligand that binds to the ligand binding domain of the membrane-associated estrogen receptor thereby inhibiting membrane-associated estrogen receptor signalling. In a preferred embodiment, the inhibitory ligand may not cross the cell membrane and in this way limits the non or less specific effects of ligands which cross this barrier. In a specific embodiment of this method, the inhibitory ligand is selected from the group consisting of monoclonal antibodies Ab 1 and Ab 2 (see FIG. 14A).

Yet another embodiment of the invention consists of inhibiting the signalling of a membrane-associated estrogen receptor with an inhibitory ligand and further contacting the cell with an anti-HER-2 immunoglobulin polypeptide under conditions which allow the anti-HER-2 immunoglobulin polypeptide to bind to HER-2 on the surfaces of the cancer cell to a degree sufficient to inhibit the growth of the cancer cell. In yet another embodiment, one can further treat the cell with an anti-HER-1/EGF receptor immunoglobulin polypeptide that binds to EGF receptor on the surfaces of the cancer cell or with conventional cancer therapy selected from the group consisting of surgical excision and chemotherapy.

Yet another embodiment of the invention is an assay for identifying and/or characterizing a ligand that can inhibit membrane-associated estrogen receptor signalling. In particular, the disclosure provided herein identifies a set of standard ligands which provide parameter means for identifying and assessing novel molecules. Typically this aspect of the invention provides a process for identifying and/or assessing the ability of a novel molecule to inhibit membrane-associated estrogen receptor signalling in a comparative assay that compares the novel molecule's measured activity with that of one of the inhibitory ligand identified herein (e.g. monoclonal antibodies Ab 1 and Ab 2).

A specific embodiment of the invention consists of a method for identifying a compound which inhibits membrane-associated estrogen receptor signalling comprising the steps of contacting a membrane-associated estrogen receptor with a test compound, and determining whether said compound inhibits the activation and/or signalling of the membrane-associated estrogen receptor. Typically, a molecule that inhibits the activation of membrane-associated estrogen receptor is useful for inhibiting membrane-associated estrogen receptor associated cellular activities. Also included in this embodiment is a product identified by such methods.

There are a variety of art accepted methods for measuring the inhibition of a membrane-associated estrogen receptor, a number of which are described herein. Typical preferred embodiments described herein include the inhibition of the estrogen-induced growth of MCF-7 breast cancer cells in vitro as described in Example 8 and the observing an inhibition in the growth of MCF-7 breast cancer xenografts in nude mice as described in Example 9. In another embodiment, inhibition of membrane-associated estrogen receptor mediated signaling is measured by observing an inhibition in the induction of ERK/MAP kinase activation. In a related embodiment, ligand binding to the membrane-associated estrogen receptor is measured generally by a radioligand binding assay.

The invention disclosed herein also provides an assay for identifying and/or assessing other membrane-associated estrogen receptor inhibitory ligands, by, for example, providing a set of standard inhibitory ligands which provide parameter means for identifying and assessing novel molecules. Typically this aspect of the invention provides a process for identifying and/or assessing the ability of a novel molecule to bind to membrane-associated estrogen receptor in a comparative assay that compares the novel molecule's measured activity with that of one of the inhibitory ligands identified herein. In this context, a number of analytical assays for analyzing ligand-receptor interactions are well known in the art and include for example the growth inhibition and activation assays discussed in the Examples below. Also included in this embodiment is a product identified by this process. A related method for identifying a compound which inhibits membrane-associated estrogen receptor signalling comprising the steps of contacting a membrane-associated estrogen receptor with a test compound, determining whether said compound binds to said membrane-associated estrogen receptor; and if the compound binds to membrane-associated estrogen receptor, determining whether said compound inhibits membrane-associated estrogen receptor activation and/or signaling. Also included in this embodiment is a product identified by such methods.

The methods for inhibiting the growth of a breast cancer cell which expresses the estrogen receptor on its cell membrane by contacting the cell with an amount of anti-estrogen receptor immunoglobulin polypeptide sufficient to inhibit cell growth have a number of a advantages over existing methods. For example, because the anti-estrogen receptor polypeptides of the present invention will not recognize cells which do not express this molecule at their surface membrane, the methods disclosed herein have a greater specificity that methods which utilize the less specific art accepted compositions traditionally used in the treatment of breast cancer. Moreover, the toxicity of these lineage specific, extracellular molecules is less than the toxicity observed with other, less specific molecules used in the treatment of cancers.

As noted above, the present invention includes methods of diagnosing and treating an individual suspected of suffering from breast cancer comprising the steps of administering to said individual a pharmaceutical composition comprising a pharmaceutically acceptable carrier or diluent, and a diagnostically or therapeutically effective amount of a compound consisting of an anti-estrogen receptor immunoglobulin polypeptide.

The methods disclosed herein include a number of different embodiments. In an illustrative embodiment, the invention consists of a method for inhibiting the growth of a breast cancer cell which expresses the estrogen receptor in association with its surface membrane by contacting the cell with an amount of anti-estrogen receptor immunoglobulin polypeptide sufficient to inhibit cell growth. In preferred embodiments of this method, the anti-estrogen receptor immunoglobulin polypeptide recognizes and binds the ligand binding domain of the estrogen receptor. In preferred embodiments of this method, the anti-estrogen receptor immunoglobulin polypeptide is selected from the group consisting of monoclonal antibodies Ab 1 and Ab 2.

A related embodiment of the invention consists of a method for treating a mammalian cancer cell which expresses estrogen receptor in association with its plasma membrane by contacting the cancer cell with the anti-estrogen receptor immunoglobulin polypeptide under conditions which allow the anti-estrogen receptor immunoglobulin polypeptide to bind to the estrogen receptor associated with the plasma membranes of the cancer cell to a degree sufficient to inhibit the growth of the cancer cell. In preferred embodiments of this method, the anti-estrogen receptor immunoglobulin polypeptide is selected from the group consisting of monoclonal antibodies Ab 1 and Ab 2.

Yet another related embodiment of the invention consists of a method of inhibiting the growth of a breast cancer cell having disregulated cell growth comprising the steps of confirming the presence of a estrogen receptor associated with the surface membrane of the breast cancer cell, providing a estrogen receptor immunoglobulin polypeptide specific for an epitope within the ligand binding domain of the estrogen receptor, the anti-estrogen receptor immunoglobulin polypeptide being selected to produce inhibition of breast cancer cell growth and then contacting the cell with the anti-estrogen receptor immunoglobulin polypeptide under conditions which allow the anti-estrogen receptor immunoglobulin polypeptide to bind to the estrogen receptor associated with the surface membrane of the breast cancer cell to a degree sufficient to inhibit the growth of the breast cancer cell. The confirmatory step is useful in contexts where the expression of membrane estrogen receptor is in question. Preferably, an anti-estrogen receptor immunoglobulin polypeptide is used to confirm the presence of this molecule, for example one that has the same specificity as the molecule used in the therapeutic method. In preferred embodiments of this method, the anti-estrogen receptor immunoglobulin polypeptide is selected from the group consisting of monoclonal antibodies Ab 1 and Ab 2.

In a variation of the methods utilizing anti-estrogen receptor immunoglobulin polypeptides discussed above, one can further contact the cell with a second therapeutic molecule used in the treatment of cancers, for example an anti-HER-2 immunoglobulin polypeptide or an anti-HER-1/EGF receptor immunoglobulin polypeptide, under conditions which inhibit the growth of the cancer cell. In yet another variation of the methods utilizing anti-estrogen receptor immunoglobulin polypeptides discussed above, one can further treat the cancer cell with a conventional therapy selected from the group consisting of surgical excision and chemotherapy.

Another embodiment of the invention consists of a method of inhibiting the activation of a cellular process that is induced via a membrane-associated estrogen receptor by contacting a cell with an anti-estrogen receptor immunoglobulin polypeptide under conditions which allow the anti-estrogen receptor immunoglobulin polypeptide to bind to estrogen receptor associated with the surface membrane of the cell to a degree sufficient to inhibit the activation of a cellular process that is induced via the membrane-associated estrogen receptor. This embodiment is consistent with observations that the pharmacology of estrogen receptors is complex and that subtle differences in their structure, as well as their cellular milieu in which they are acting (e.g. the cellular membrane), can have marked effects on specific responses within the cell (see e.g. Katzenellenboogen et al., Breast Cancer Research and Treatment 44: 23-38 (1997).

Another embodiment of the invention disclosed herein includes an injectable pharmaceutical composition for treatment of a mammalian cancer tumor having cells which express estrogen receptor associated with their cell membranes consisting of an anti-estrogen receptor immunoglobulin polypeptide specific to an epitope on a ligand binding domain of the estrogen receptor; the anti-estrogen receptor immunoglobulin polypeptide being selected for its ability to inhibit tumor growth; and a pharmaceutically acceptable injection vehicle. Preferably, the anti-estrogen receptor immunoglobulin polypeptide is selected from the group consisting of monoclonal antibodies Ab 1 and Ab 2.

Yet another embodiment of the invention disclosed herein includes a kit for use in methods for inhibiting the growth of breast tumor cells which express an estrogen receptor comprising a container, a composition contained within the container, wherein the composition includes an anti-estrogen receptor immunoglobulin polypeptide and instructions for using the anti-estrogen receptor immunoglobulin polypeptide in vivo or in vitro. Preferably, the anti-estrogen receptor immunoglobulin polypeptide is selected from the group consisting of monoclonal antibodies Ab 1 and Ab 2.

Yet another embodiment of the invention disclosed herein includes a method of radioimaging metastasized breast cancer cells comprising the steps of first administering to an individual suspected of having metastasized breast cancer cells, a pharmaceutical composition that consists of a pharmaceutically acceptable carrier or diluent, and coupled compound that comprises an anti-estrogen receptor immunoglobulin polypeptide and a radioactive active moiety wherein the coupled compound is present in an amount effective for diagnostic use in humans suffering from breast cancer and then detecting the localization and accumulation of radioactivity in the individual's body.

Various embodiments and aspects of the invention are described in detail below.

III. Molecules for Use in the Methods of the Invention

Immunoglobulin Polypeptides

The anti-estrogen receptor immunoglobulin polypeptides of the present invention may be obtained through a variety of sources including commercial suppliers. Alternatively, the anti-estrogen receptor immunoglobulin polypeptides of the present invention may be prepared by immunizing an animal with a purified or partially purified human estrogen receptor. The animals immunized can be any one of a variety of species which are capable of immunologically recognizing epitopes characteristic of the human estrogen receptor, such as murine, porcine, equine, etc.

Monoclonal antibodies of the invention may be prepared by immortalizing nucleic acid sequences which encode immunoglobulin polypeptides or portions thereof that bind specifically to antigenic determinants characteristic of the human estrogen receptor. The immortalization process can be carried out by hybridoma fusion techniques, by viral transformation of antibody-producing lymphocytes, recombinant DNA techniques, or by techniques that combine cell fusion, viral transformation and/or recombinant DNA methodologies. According to one aspect of the invention, cells producing human anti-estrogen receptor monoclonal antibodies are immortalized using, e.g., Epstein-Barr virus (EBV) transformation techniques. For example, B lymphocytes derived from peripheral blood, bone marrow, lymph nodes, tonsils, etc. of patients, preferably those immunized with the estrogen receptor or portions thereof, are immortalized using EBV according to methods such as those described in U.S. Pat. No. 4,464,465, and Chan et al., J. Immunol. 136:106 (1986), which are incorporated herein by reference.

Human anti-estrogen receptor monoclonal antibodies can also be prepared by a variety of other ways, e.g., using a combination of EBV or other viral transformation and hybridoma fusion techniques. For instance, the hybridomas can be created by fusing stimulated B cells, obtained from a individual immunized with the estrogen receptor or a portion thereof, with a mouse/human heterohybrid fusion partner, a variety of which have been described. See, e.g., U.S. Pat. No. 4,624,921 and James and Bell, J. Immunol. Meths. 100:5-40 (1987), which are incorporated herein by reference. A mouse/human fusion partner can be constructed by fusing human lymphocytes stimulated or transformed by EBV with readily available mouse myeloma lines such as NS-1 or P3NS-1, in the presence of, e.g., polyethylene glycol.

The hybridomas or lymphoblastoid cells which secrete antibody of interest can be identified by screening culture supernatants for antibody that binds to the estrogen receptor. More preferably, a screening assay may be employed to detect those antibodies which inhibit, for example, estrogen-mediated mitogenesis. Cells which possess the desired activity are cloned and subcloned in accordance with conventional techniques and monitored until stable, immortalized lines producing the anti-estrogen receptor monoclonal antibody of interest are identified. By monoclonal antibody is meant an antibody produced by a clonal, immortalized cell line separate from cells producing antibodies with a different antigen binding specificity. Thus, such monoclonal antibodies are produced isolated from other monoclonal antibodies and, accordingly, in substantially pure form (relative to other antibodies) and at a concentration generally greater than normally occurring in sera from the animal species which serves as a B cell source.

Alternatively, one can isolate DNA sequences which encode a human anti-estrogen receptor immunoglobulin polypeptide or portions thereof that specifically bind to the estrogen receptor, or a specific domain of the estrogen receptor (such as the ligand binding domain) by screening a DNA library from human B cells according to a general protocol as disclosed by Huse et al., Science 246:1275-1281 (1989), incorporated herein by reference, and then cloning and amplifying the sequences which encode the anti-estrogen receptor antibodies (or binding fragments) of the desired specificity.

The immunoglobulins may then be produced by introducing an expression vector containing the appropriate immunoglobulin gene, or portion thereof, into an appropriate host cell. The host cell line is then maintained under conditions suitable for high level expression of the immunoglobulin nucleotide sequences, and, as desired, the collection and purification of the light chains, heavy chains, light/heavy chain dimers or intact antibodies, binding fragments or other immunoglobulin forms may follow. Suitable host cells include microorganisms, but mammalian or insect tissue cell culture may be preferable for producing the monoclonal antibody of the present invention (see, E. Winnacker, “From Genes to Clones,” VCH Publishers, N.Y., N.Y. (1987), which is incorporated herein by reference). A number of suitable mammalian host cell lines capable of secreting intact immunoglobulins have been developed in the art, and include the Chinese hamster ovary (CHO) cell line, but preferably hybridomas or transformed B-cells will be used. Bacterial phage or yeast systems may also be employed.

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), incorporated herein by reference). Once purified, partially or to homogeneity as desired, the polypeptides may then be used therapeutically (including extracorporeally) or in developing and performing assay procedures, immunofluorescent stainings, and the like. (See, generally, Immunological Methods, Vols. I and II, Lefkovits and Pernis, eds., Academic Press, New York, N.Y. (1979 and 1981), which are incorporated herein by reference).

The immunoglobulin polypeptides produced according to the present invention may be of the IgG, IgM, IgA or IgD isotype, and may further be any of the appropriate subclasses thereof, such as, e.g., IgG₁, IgG₂, IgG₃, or IgG₄. Using recombinant DNA techniques, “class-switching” of the isolated immunoglobulin polypeptides may be readily accomplished. In this method genes encoding the constant regions which determine the isotype of the immunoglobulin molecule of interest are replaced by genes encoding a desired isotype or subclass, as generally described in European patent publication EP 314,161, incorporated herein by reference. Class-switched immunoglobulins may also be isolated by selecting cells which have undergone spontaneous switching using selection methods known in the art.

The administration to humans of immunoglobulin polypeptides which are substantially non-human may elicit anti-antibody responses. Thus, it may be desirable to prepare chimeric anti-estrogen receptor immunoglobulin polypeptides of the present invention which are substantially human. By “substantially human” is meant an antibody or binding fragment thereof comprised of amino acid sequences which are at least about 50% human in origin, at least about 70 to 80% more preferred, and about 95-99% or more human most preferred, particularly for repeated administrations over a prolonged period as may be necessary to treat established estrogen-mediated cell proliferation disorders.

As noted above, chimeric antibodies or chimeric immunoglobulin polypeptides that specifically bind to the human estrogen receptor and thus inhibit binding of estrogen to the receptor are also within the scope of the present invention. A typical therapeutic chimeric antibody would be a hybrid protein consisting of the variable (V) or antigen-binding domain from a mouse immunoglobulin specific for a human estrogen receptor antigenic determinant, and the constant (C) or effector domain from a human immunoglobulin, although domains from other mammalian species may be used for both variable and constant domains. As used herein, the term “chimeric antibody” also refers to antibodies coded for by immunoglobulin genes in which only the complementarity determining regions (CDR's) are transferred from the immunoglobulin that specifically recognizes the antigenic determinants, the remainder of the immunoglobulin gene being derived from a human (or other mammalian, as desired) immunoglobulin gene. This type of chimeric antibody is referred to as a “humanized” (in the case of a human immunoglobulin gene being used) antibody. The hypervariable regions of the variable domains of the anti-estrogen receptor immunoglobulin polypeptides comprise a related aspect of the invention. The hypervariable regions, or CDRs, in conjunction with the framework regions (those portions of immunoglobulin light and heavy chain variable regions that are relatively conserved among different immunoglobulins in a single species), enable the anti-estrogen receptor immunoglobulin polypeptides to recognize and thus bind to the human estrogen receptor. The hypervariable regions can be cloned and sequenced. Once identified, these regions that confer specific recognition of the estrogen receptor can then be cloned into a vector for expression in a host as part of another immunoglobulin molecule or as a fusion protein, e.g., a carrier molecule which functions to enhance immunogenicity of the cloned idiotope

As used herein, human antibody is meant to include antibodies of entirely human origin as well as those which are substantially human, unless the context indicates otherwise. As the generation of human anti-estrogen receptor monoclonal antibodies may be difficult with conventional immortalization techniques, it may be desirable to first make non-human antibodies and then transfer via recombinant DNA techniques the antigen binding regions of the non-human antibodies, e.g., the Fab, complementarity determining regions (CDRs) or hypervariable regions, to human constant regions (Fc) or framework regions as appropriate to produce substantially human molecules. Such methods are generally known in the art and are described in, for example, U.S. Pat. No. 4,816,397, PCT publication WO 90/07861, and EP publications 173494 and 239400, wherein each is incorporated herein by reference.

Human constant region DNA sequences can be isolated in accordance with well known procedures from a variety of human cells, but preferably from immortalized B-cells. The variable regions or CDRs for producing the chimeric immunoglobulins of the present invention may be similarly derived from monoclonal antibodies capable of binding to the human estrogen receptor, and will be produced in any convenient mammalian system, including, mice, rats, rabbits, human cell lines, or other vertebrates capable of producing antibodies by well known methods. Variable regions or CDRs may be produced synthetically, by standard recombinant methods including polymerase chain reaction (PCR) or through phage-display libraries. For phage display methods, see for example, McCafferty et al. Nature 348:552-554 (1990); Clackson et al. Nature 352:624-628; and Marks et al. Biotechnology 11:1145-1149 (1993).

Completely human antibodies can also be produced in transgenic animals. The desired human immunoglobulin genes or gene segments can be isolated, for example by PCR from human B cells, the DNA cloned into appropriate vectors for expression in eukaryotic cells and the cloned DNA introduced into animals to produce transgenics. Animals suitable for the production of transgenics expressing human immunoglobulins include mice, rats, rabbits and pigs with rodents being preferred. Mice and other animals for the preparation of transgenics that express human immunoglobulins should preferably have one or more of their endogenous immunoglobulin loci inactivated or “knocked-out” to facilitate identification and isolation of the human antibodies (See e.g., Lonberg, et al. Nature 368:856-859 (1994)).

In addition to the chimeric and “humanized” immunoglobulins specifically described herein, other substantially identical modified immunoglobulins can be readily designed and manufactured utilizing various recombinant DNA techniques well known to those skilled in the art. In general, modifications of the genes may be readily accomplished by a variety of well-known techniques, such as PCR and site-directed mutagenesis (see, Gillman and Smith, Gene 8:81-97 (1979) and S. Roberts et al., Nature 328:731-734 (1987), both of which are incorporated herein by reference).

Alternatively, polypeptide fragments comprising only a portion of the primary immunoglobulin structure may be produced. For example, it may be desirable to produce immunoglobulin polypeptide fragments that possess one or more immunoglobulin activities in addition to, or other than, antigen recognition (e.g., complement fixation).

Single-chain antibodies, in which genes for a heavy chain and a light chain are combined into a single coding sequence, may also be used in the methods disclosed herein. Immunoglobulin polypeptide also encompasses a truncated immunoglobulin chain, for example, a chain containing less constant region domains than in the native polypeptide. Such truncated polypeptides can be produced by standard methods such as introducing a stop codon into the gene sequence 5′ of the domain sequences to be deleted. The truncated polypeptides can then be assembled into truncated antibodies.

Those skilled in the art appreciate that “anti-idiotypic” antibodies can be produced by using a specific immunoglobulin as an immunogen in accordance with standard techniques. For example, infection or immunization with a estrogen receptor polypeptide, or fragment thereof, induces a neutralizing immunoglobulin, which has on its Fab variable region combining site an image of the estrogen receptor polypeptide that is unique to that particular immunoglobulin, i.e., an idiotype. Immunization with such an anti-estrogen immunoglobulin polypeptide induces an anti-idiotype antibody, which has a conformation at its combining site that mimics the structure of the original estrogen receptor antigen. These anti-idiotype antibodies may therefore be used to treat estrogen-mediated diseases. The anti-estrogen receptor immunoglobulin polypeptides of the invention find utility in therapeutic and diagnostic methods and compositions. For therapeutic uses, anti-estrogen receptor immunoglobulin polypeptides are used as a soluble ligand for human estrogen receptor, masking the receptor or otherwise inhibiting estrogen molecules from binding to the receptor, and thereby inhibiting the undesired cell migration and proliferation.

Antibodies as used herein also include bispecific antibodies which can be produced such as by the methods described in the following references: Glennie et al. J. Immunol. 139:2367-2375 (1987); Segal et al. Biologic Therapy of Cancer Therapy of Cancer Updates 2(4):1-12 (1992); and Shalaby et al. J. Exp. Med. 175:217-225 (1992).

The anti-estrogen receptor immunoglobulin polypeptides of the invention will generally be used intact, or as immunogenic fragments, such as F_{V}, Fab, F(ab′), or F(ab′)₂ fragments. The fragments may be obtained from antibodies by conventional techniques, such as by proteolytic digestion of the antibody using, e.g., pepsin or papain, or by recombinant DNA techniques in which a gene or portion thereof encoding the desired fragment is cloned or synthesized, and expressed in a variety of hosts.

Coupling Molecules

As noted above, when breast cancer cells metastasize, the metastasized cancer cells continue to produce and display the estrogen receptor. In this context, estrogen receptors permit some specificity for localizing therapeutic and diagnostic agents that are coupled to anti-estrogen receptor immunoglobulin polypeptides that target breast cancer cells.

One having ordinary skill in the art can readily identify individuals suspected of suffering from breast cancer and metastasized breast cells. In those individuals diagnosed with breast cancer, it is standard therapy to suspect metastasis and aggressively attempt to eradicate metastasized cells. The present invention provides pharmaceutical compositions and methods for imaging and thereby will more definitively diagnose metastasis. Further, the present invention provides pharmaceutical compositions, comprising therapeutic agents and methods for specifically targeting and eliminating breast cancer cells.

Immunoglobulin polypeptides, in whole or in part, may be utilized alone or coupled with active molecules such as the functional regions from other genes (e.g., enzymes), or other molecules such as toxins, labels and targeting moieties to produce fusion proteins (e.g., “immunotoxins”) having novel properties. In these cases of gene fusion, the two components are present within the same polypeptide chain. Alternatively, the immunoglobulin or fragment thereof may be chemically bonded to the toxin or label by any of a variety of well-known chemical procedures. For example, when the label or cytotoxic agent is a protein and the second component is an intact immunoglobulin, the linkage may be by way of heterobifunctional cross-linkers, e.g., SPDP, carbodiimide, glutaraldehyde, or the like.

Anti-estrogen receptor immunoglobulin polypeptides are conjugated to active agents by a variety of well-known techniques readily performed without undue experimentation by those having ordinary skill in the art. The technique used to conjugate the anti-estrogen receptor immunoglobulin polypeptide to the active agent is dependent upon the molecular nature of the anti-estrogen receptor immunoglobulin polypeptide and the active agent. After the anti-estrogen receptor immunoglobulin polypeptide and the active agent are conjugated to form a single molecule, assays may be performed to ensure that the conjugated molecule retains the activities of the moieties. The estrogen receptor binding assay described above may be performed using the conjugated compound to test whether it is capable of binding to the estrogen receptor. Similarly, the activity of the active moiety may be tested using various assays for each respective type of active agent. Radionuclides retain their activity, i.e. their radioactivity, irrespective of conjugation. With respect to active agents which are toxins, drugs and targeting agents, standard assays to demonstrate the activity of unconjugated forms of these compounds may be used to confirm that the activity has been retained.

Conjugation may be accomplished directly between the anti-estrogen receptor immunoglobulin polypeptide and the active agent or linking, intermediate molecular groups may be provided between the anti-estrogen receptor immunoglobulin polypeptide and the active agent. Crosslinkers are particularly useful to facilitate conjugation by providing attachment sites for each moiety. Crosslinkers may include additional molecular groups which serve as spacers to separate the moieties from each other to prevent either from interfering with the activity of the other.

According to the present invention, the active moieties may be an imaging agent. Imaging agents are useful diagnostic procedures as well as the procedures used to identify the location of metastasized cells. Imaging can be performed by many procedures well-known to those having ordinary skill in the art and the appropriate imaging agent useful in such procedures may be conjugated to an anti-estrogen receptor immunoglobulin polypeptide by well-known means. Imaging can be performed, for example, by radioscintigraphy, nuclear magnetic resonance imaging (MRI) or computed tomography (CT scan). The most commonly employed radionuclide imaging agents include radioactive iodine and indium. Imaging by CT scan may employ a heavy metal such as iron chelates. MRI scanning may employ chelates of gadolinium or manganese. Additionally, positron emission tomography (PET) is possible using positron emitters of oxygen, nitrogen, iron, carbon, or gallium. Examples of radionuclides useful in imaging procedures include: ⁴³K, ⁵²Fe, ⁵⁷Co, ⁶⁷Cu, ⁶⁷Ga, ⁶⁸Ga, ⁷⁷Br, ⁸¹Rb, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi.

According to the present invention, the active moieties may be a therapeutic agent such as a chemotherapeutic drug. One having ordinary skill in the art may conjugate an anti-estrogen receptor immunoglobulin polypeptide peptide to a chemotherapeutic drug using well-known techniques. For example, Magerstadt, M. Antibody Conjugates and Malignant Disease. (1991) CRC Press, Boca Raton, USA, pp. 110-152) which is incorporated herein by reference, teaches the conjugation of various cytostatic drugs to amino acids of antibodies. Such reactions may be applied to conjugate chemotherapeutic drugs to anti-estrogen receptor immunoglobulin polypeptides, including estrogen receptor binding peptides, with an appropriate linker. Anti-estrogen receptor immunoglobulin polypeptides which have a free amino group such as estrogen receptor binding peptides may be conjugated to active agents at that group. Most of the chemotherapeutic agents currently in use in treating cancer possess functional groups that are amenable to chemical crosslinking directly with proteins. For example, free amino groups are available or methotrexate, doxorubicin, daunorubicin, cytosinarabinoside, cis-platin, vindesine, mitomycin and bleomycin while free carboxylic acid groups are available on methotrexate, melphalan, and chlorambucil. These functional groups, that is free amino and carboxylic acids, are targets for a variety of homobifunctional and heterobifunctional chemical crosslinking agents which can crosslink these drugs directly to the single free amino group of estrogen. For example, one procedure for crosslinking anti-estrogen receptor immunoglobulin polypeptides which have a free amino group such as estrogen receptor binding peptides, for example anti-estrogen receptor immunoglobulin polypeptides such as monoclonal antibodies Ab 311 and Ab 10, to active agents which have a free amino group such as methotrexate, doxorubicin, daunorubicin, cytosinarabinoside, cisplatin, vindesine, mitomycin and bleomycin, or alkaline phosphatase, or protein- or peptide-based toxin employs homobifunctional succinimidyl esters, preferably with carbon chain spacers such as disuccinimidyl suberate (Pierce Co, Rockford, Ill.). In the event that a cleavable conjugated compound is required, the same protocol would be employed utilizing 3,3′-dithiobis (sulfosuccinimidylpropionate; Pierce Co.).

In order to conjugate an anti-estrogen receptor immunoglobulin polypeptide to a peptide-based active agent such as a toxin, the anti-estrogen receptor immunoglobulin polypeptide and the toxin may be produced as a single, fusion protein either by standard peptide synthesis or recombinant DNA technology, both of which can be routinely performed by those having ordinary skill in the art. Alternatively, two peptides, the anti-estrogen receptor immunoglobulin polypeptide and the peptide-based toxin may be produced and/or isolated as separate peptides and conjugated using crosslinkers. As with conjugated compositions that contain chemotherapeutic drugs, conjugation of estrogen receptor binding peptides and toxins can exploit the ability to modify the single free amino group of an estrogen receptor binding peptide while preserving the receptor-binding function of this molecule.

One having ordinary skill in the art may conjugate an anti-estrogen receptor immunoglobulin polypeptide to a radionuclide using well-known techniques. For example, Magerstadt, M. (1991) Antibody Conjugates And Malignant Disease, CRC Press, Boca Raton, Fla., and Barchel, S. W. and Rhodes, B. H., (1983) Radioimaging and Radiotherapy, Elsevier, NY, N.Y., each of which is incorporated herein by reference, teach the conjugation of various therapeutic and diagnostic radionuclides to amino acids of antibodies. Such reactions may be applied to conjugate radionuclides to anti-estrogen receptor immunoglobulin polypeptides or to anti-estrogen receptor immunoglobulin polypeptides including anti-estrogen receptor immunoglobulin polypeptides with an appropriate linker. Suitable labels include, for example, radionuclides, enzymes, substrates, cofactors, inhibitors, fluorescers, chemiluminescers, magnetic particles. See, for examples of patents teaching the use of such labels, U.S. Pat. Nos. 3,817,837; 3,850,752; 3,939,350; 3,996,345; 4,277,437; 4,275,149; and 4,366,241, all of which are incorporated by reference.

Immunotoxins, including single chain molecules for use in the disclosed methods, may be produced by recombinant means. Production of various immunotoxins is well-known with the art, and methods can be found, for example in “Monoclonal Antibody-Toxin Conjugates: Aiming the Magic Bullet,” Thorpe et al, Monoclonal Antibodies in Clinical Medicine, Academic Press, pp. 168-190 (1982); E. Vitetta, Science (1987) 238:1098-1104; and G. Winter and C. Milstein, Nature (1991) 349:293-299; all incorporated herein by reference. A variety of cytotoxic agents are suitable for use in immunotoxins. Cytotoxic agents can include radionuclides, such as Iodine-131, Yttrium-90, Rhenium-188, and Bismuth-212; a number of chemotherapeutic drugs, such as vindesine, methotrexate, adriamycin, and cisplatinum; and cytotoxic proteins such as ribosomal inhibiting proteins like pokeweed antiviral protein, Pseudomonas exotoxin A, ricin, diphtheria toxin, ricin A chain, etc., or an agent active at the cell surface, such as the phospholipase enzymes (e.g., phospholipase C). (See, generally, “Chimeric Toxins,” Olsnes and Pihl, Pharmac. Ther., 15:355-381 (1981), and “Monoclonal Antibodies for Cancer Detection and Therapy,” eds. Baldwin and Byers, pp. 159-179, 224-266, Academic Press (1985), both of which are incorporated herein by reference).

The present invention provides pharmaceutical compositions that comprise the compounds of the invention and pharmaceutically acceptable carriers or diluents. The pharmaceutical composition of the present invention may be formulated by one having ordinary skill in the art. Suitable pharmaceutical carriers are described in Remington's Pharmaceutical Sciences, A. Osol, a standard reference text in this field, which is incorporated herein by reference. In carrying out methods of the present invention, conjugated compounds of the present invention can be used alone or in combination with other diagnostic, therapeutic or additional agents. Such additional agents include excipients such as coloring, stabilizing agents, osmotic agents and antibacterial agents.

Other Inhibitory Molecules

Other inhibitory molecules of the present invention, such as anti-estrogen receptor antagonists (ICI 182,780) or signaling inhibitors of membrane-associated estrogen receptor-induced signal transduction pathways (including MAP kinase, PI3K/Akt kinase, adenylate cyclase and G-protein coupled, and nitric oxide signaling pathways), may be obtained through a variety of sources including commercial suppliers. Alternatively, anti-membrane estrogen receptor reagents of the present invention may be prepared by the design and synthesis of selective membrane-associated estrogen receptor modulators of steroidal or non-steroidal nature (see Pietras et al., Proc. Am. Assoc. Cancer Res. 40: 637, 1999).

IV. Uses of the Invention

As illustrated below, the methods for utilizing immunoglobulin polypeptides and other inhibitory molecules of the present invention have uses in therapeutic, diagnostic and other applications.

A. General Uses

A wide variety of pathological syndromes which involve estrogen mediated signalling are described in the art (see e.g. U.S. Pat. Nos. 6,028,064, 5,585,405, 5,877,219 and 5,395,842). In this context, the methods of the invention provided herein are shown have a number of different utilities. In particular, the identification of the one or more ligands which inhibit the activity a receptor that is active in a number of distinct subcellular environments including. the membrane and cell nucleus (particularly one shown to be associated with pathological conditions) is a crucial element in many protocols employed in the art for a variety of purposes. Such uses include for example, the determination of membrane-associated estrogen receptor ligand concentrations, the development of membrane-associated estrogen receptor ligand agonists and antagonists and the characterization of the physiological roles of these ligands and their receptors. Consequently, a wide variety of protocols based on methods pertaining to such ligand-receptor interactions are described for these purposes (see, e.g., U.S. Pat. No. 5,871,909, U.S. Pat. No. 5,030,576, U.S. Pat. No. 6,110,737 and U.S. Pat. No. 6,040,290). In this context, the disclosure provided herein allows a comprehensive characterization of the role of membrane-associated estrogen receptor in normal physiological processes as well as the pathological conditions associated with membrane-associated estrogen receptor signalling.

B. Diagnostic Methods of the Invention

As noted above, one aspect of the present invention relates to a method of detecting metastasized breast cancer cells in an individual suspected of suffering from metastasized breast cancer by radioimaging. Such individuals may be diagnosed as suffering from metastasized breast cancer and the metastasized breast cancer cells may be detected by administering to the individual, preferably by intravenous administration, a pharmaceutical composition that comprises a pharmaceutically acceptable carrier or diluent and a conjugated compound that comprises an anti-estrogen receptor immunoglobulin polypeptide and an active moiety wherein the active moiety is a radioactive and detecting the presence of a localized accumulation or aggregation of radioactivity, indicating the presence of cells with membrane estrogen receptors.

In some embodiments of the present invention, the pharmaceutical composition comprises a pharmaceutically acceptable carrier or diluent and a conjugated compound that comprises an anti-estrogen receptor immunoglobulin polypeptide and an active moiety wherein the active moiety is a radioactive. In typical embodiments of the present invention, the pharmaceutical composition comprises a pharmaceutically acceptable carrier or diluent and a conjugated compound that comprises an anti-estrogen receptor immunoglobulin polypeptide and an active moiety wherein the active moiety is a radioactive agent selected from the group consisting of: ⁴³K, ⁵²Fe, ⁵⁷Co, ⁶⁷Cu, ⁶⁷Ga, ⁶⁸Ga, ⁷⁷Br, ⁸¹Rb, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi. The individual being treated may be diagnosed as having metastasized breast cancer or may be diagnosed as having localized breast cancer and may undergo the treatment proactively in the event that there is some metastasis as yet undetected. The pharmaceutical composition contains a diagnostically effective amount of the conjugated composition. A diagnostically effective amount is an amount which can be detected at a site in the body where cells with estrogen receptors are located without causing lethal side effects on the individual.

C. Therapeutic Methods of the Invention

As noted above, one aspect of the present invention relates to a method of treating individuals suspected of suffering from breast cancer. Such individuals may be treated by administering to the individual a pharmaceutical composition that comprises a pharmaceutically acceptable carrier or diluent and a anti-estrogen receptor immunoglobulin polypeptide. In one embodiment, the pharmaceutical composition includes a conjugated compound that comprises an anti-estrogen receptor immunoglobulin polypeptide and an active moiety wherein the active moiety is a therapeutic agent. In illustrative embodiments of the present invention, the pharmaceutical composition comprises a pharmaceutically acceptable carrier or diluent and a conjugated compound that comprises an anti-estrogen receptor immunoglobulin polypeptide and an active moiety wherein the active moiety is a radiostable active agent. Typically, the active moiety is selected from the group consisting of: methotrexate, doxorubicin, daunorubicin, cytosinarabinoside, etoposide, 5-4 fluorouracil, melphalan, chlorambucil, cis-platinum, vindesine, mitomycin, bleomycin, purothionin, macromomycin, 1,4-benzoquinone derivatives, trenimon, ricin, ricin A chain, Pseudomonas exotoxin, diphtheria toxin, Clostridium perfringens phospholipase C, bovine pancreatic ribonuclease, pokeweed antiviral protein, abrin, abrin A chain, cobra venom factor, gelonin, saporin, modeccin, viscumin, volkensin, alkaline phosphatase, nitroimidazole, metronidazole and misonidazole.

In some embodiments of the present invention, the pharmaceutical composition comprises a pharmaceutically acceptable carrier or diluent and a conjugated compound that comprises an anti-estrogen receptor immunoglobulin polypeptide and an active moiety wherein the active moiety is a radioactive agent. In typical embodiments of the present invention, the pharmaceutical composition comprises a pharmaceutically acceptable carrier or diluent and a conjugated compound that comprises an anti-estrogen receptor immunoglobulin polypeptide and an active moiety wherein the active moiety is a radioactive agent selected from the group consisting of: ⁴³K, ⁵²Fe, ⁵⁷Co, ⁶⁷Cu, ⁶⁷Ga, ⁶⁸Ga, ⁷⁷Br, ⁸¹Rb, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi.

The individual being treated may be diagnosed as having metastasized breast cancer or may be diagnosed as having localized breast cancer and may undergo the treatment proactively in the event that there is some metastasis as yet undetected. The pharmaceutical composition contains a therapeutically effective amount of the conjugated composition. A therapeutically effective amount is an amount which is effective to cause a cytotoxic or cytostatic effect on metastasized breast cancer cells without causing lethal side effects on the individual. Another embodiment of the present invention relates to a method of treating individuals suspected of suffering from metastasized breast cancer.

The individual being treated may be diagnosed as having metastasized breast cancer or may be diagnosed as having localized breast cancer and may undergo the treatment proactively in the event that there is some metastasis as yet undetected. The pharmaceutical composition contains a therapeutically effective amount of the coupled composition. A therapeutically effective amount is an amount which is effective to cause a cytotoxic or cytostatic effect on metastasized breast cancer cells without causing lethal side effects on the individual.

IV. Administration of Pharmaceutical Compositions

For pharmaceutical compositions, the anti-estrogen receptor immunoglobulin polypeptides of the invention as described herein can be administered to an individual having a estrogen-mediated cellular proliferation disorder. In therapeutic applications, compositions are administered to a patient in an amount sufficient to effectively block cell receptors, and thereby cure or at least partially arrest the cellular proliferation and its symptoms and/or complications. An amount adequate to accomplish this is defined as “therapeutically effective dose.” Amounts effective for this use will depend on a variety of factor, for example, the nature of the anti-estrogen receptor immunoglobulin polypeptide composition, the manner of administration, the stage and severity of the disease being treated, the weight and general state of health of the patient, and the judgment of the prescribing physician.

Generally, effective amounts of such molecules range from about 0.01 mg/kg to about 100.0 mg/kg of antibody per day, with dosages of from about 0.1 mg/kg to about 10.0 mg/kg of antibody per day being more commonly used. It must be kept in mind that the anti-estrogen receptor immunoglobulin polypeptide and peptide compositions derived therefrom may be employed in serious disease states, that is, life-threatening or potentially life threatening situations. In such cases, it is possible and may be felt desirable by the treating physician to administer substantial excesses of these compositions. Thus, human anti-estrogen receptor monoclonal antibodies or substantially human anti-estrogen receptor monoclonal antibodies of the invention are most preferred under these circumstances.

The concentration of anti-estrogen receptor immunoglobulin polypeptides of the invention in the pharmaceutical formulations can vary widely, i.e., from less than about 1%, usually at or at least about 10-15% to as much as 50% or more by weight, and will be selected primarily by fluid volumes, viscosities, etc., in accordance with the particular mode of administration selected. A typical pharmaceutical composition for intravenous infusion could be made up to contain 250 ml of sterile Ringer's solution, and 100 mg of anti-estrogen receptor immunoglobulin polypeptide. Actual methods for preparing parenterally administrable compounds will be known or apparent to those skilled in the art and are described in more detail in for example, Remington's Pharmaceutical Science, 17th ed., Mack Publishing Company, Easton, Pa. (1985), which is incorporated herein by reference. The anti-estrogen receptor immunoglobulin polypeptides and fragments thereof can also be administered via liposomes. The anti-estrogen receptor immunoglobulin polypeptides can serve to target the liposomes to particular tissues or cells displaying the human estrogen receptor. Liposomes include emulsions, foams, nicelles, insoluble monolayers, liquid crystals, phospholipid dispersions, lamellar layers and the like. In these preparations the immunoglobulin polypeptide or fragment to be delivered is incorporated as part of the liposome, alone or in conjunction with a molecule which is, for example, toxic to the target cells. A liposome suspension containing an immunoglobulin polypeptide can be administered intravenously, locally, topically, etc. in a dose which varies according to, inter alia, the manner of administration, the peptide being delivered, and the stage of disease being treated. For solid compositions of the anti-estrogen receptor immunoglobulin polypeptides of the invention, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like.

The pharmaceutical compositions of the present invention may be administered by any means that enables the coupled composition to reach the targeted cells. In some embodiments, routes of administration include those selected from the group consisting of intravenous, intraarterial, intraperitoneal, local administration into the blood supply of the organ in which the tumor resides or directly into the tumor itself. Intravenous administration is the preferred mode of administration. It may be accomplished with the aid of an infusion pump. The dosage administered varies depending upon factors such as: the nature of the active moiety; the nature of the conjugated composition; pharmacodynamic characteristics; its mode and route of administration; age, health, and weight of the recipient; nature and extent of symptoms; kind of concurrent treatment and frequency of treatment.

Single or multiple administrations of the compositions can be carried out with dose levels and pattern being selected by the treating physician. In any event, the pharmaceutical formulations should provide a quantity of anti-estrogen receptor immunoglobulin polypeptide of the invention sufficient to effectively treat the patient. Administration should begin at the first indication of undesirable cellular proliferation or shortly after diagnosis, and continue until symptoms are substantially abated and for a period thereafter. In well established cases of disease, loading doses followed by maintenance doses will be required. The pharmaceutical compositions for therapeutic treatment are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration which comprise a solution of the anti-estrogen receptor immunoglobulin polypeptide dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

V. Kits of the Invention

For use in the diagnostic and therapeutic applications described or suggested above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise an anti-estrogen receptor immunoglobulin polypeptide that is or can be coupled to an active moiety.

The kits of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The present invention is further detailed in the following Examples, which are offered by way of illustration and are not intended to limit the invention in any manner. All patent and literature references cited in the present specification are hereby incorporated by reference in their entirety.

### EXAMPLES

### Example 1

### General Materials and Methods

Cell Culture and Assay of Cell Proliferation In Vitro

MCF-7 human breast cancer cells (ATCC) were maintained in RPMI 1640 medium with 10% fetal bovine serum (FBS). For estrogen-free conditions, medium was changed 48 h before experiments to phenol-red free RPMI 1640 with 1% dextran-coated, charcoal-treated (DCC) FBS (3). In experiments using 17β-estradiol 17-hemisuccinate covalently linked to bovine serum albumin (E₂β-BSA, Steraloids, Newport, RI, USA), aliquots of E₂β-BSA were preabsorbed with DCC to remove free steroid by established methods (94).

To assess effects of ER antibodies on cell proliferation in selected experiments, cells were first incubated with antibodies directed against different domains of ER-α: Ab1, against ligand-binding domain (LBD) from amino acids 495-595 (Upstate Biotechnology, Lake Placid, N.Y., USA); Ab2, against LBD, from amino acids 302-595 (Neomarkers, Fremont, Calif., USA) and Ab3 against hinge-region, HSP 90- and DNA-binding domains, from amino acids 280-335 (NeoMarkers). Incubation with antibodies for 2 h was followed by addition of either 10 nM E₂β, 100 nM E₂α (17α-estradiol, Steraloids), 0.5 μM E₂α-BSA (6 keto-17α-estradiol 6-(o-carboximethyloxime:BSA, Steraloids) or 0.5 μM E₂β-BSA for 10 min. After 72 h, cells were counted to estimate rates of cell proliferation, using data from 4 independent experiments.

Cell Homogenization and Subcellular Fractionation

Cell fractionation was done as before(47,48). In brief, cells were harvested with ice-cold Versene in the presence of protease inhibitors, then homogenized using a Dounce homogenizer. Whole homogenate (H) was filtered through nylon mesh and centrifuged at 1000×g for 10 min to yield crude nuclear (N) and post-nuclear supernate fractions. The N fraction was resuspended in 31% sucrose in buffer, loaded on top of a discontinuous sucrose density-gradient and centrifuged at 67,000 g for 2 h. Plasma membranes occurred predominantly at ρ=1.13-1.16 (PM) (47). The postnuclear supernatant was centrifuged at 15,000 g for 30 min, with the resulting pellet representing the mitochondria-lysosome fraction (ML). The supernate was centrifuged at 105,000 g for 1 h to yield the microsomal pellet fraction (Ms) and the soluble cytosol fraction (S). Extracts from cell membranes were solubilized as before (93). Protein was quantitated using the BCA-200 Protein Assay Kit (Pierce, Rockford, Ill., USA).

Analyses of Enzyme Activity, DNA and [³H] Estradiol-17β Binding Assay in Subcellular Fractions

Activity of 5′-nucleotidase (EC 3.1.3.5) was determined by established methods, with specific activities given as nmol/min/mg (47,48). Activity of lactate dehydrogenase (LDH) was assessed as before (100). Relative specific activity represents the specific activity of enzyme in a given fraction in relation to that in homogenate. DNA was determined by established methods (48). Specific E₂β binding was assessed in cell fractions using [2,4,6,7,16,17-³H] estradiol-17β (NEN, Boston, Mass., USA) as reported previously (47,48).

Determination of p44/42 MAPK and Akt Kinase Activity

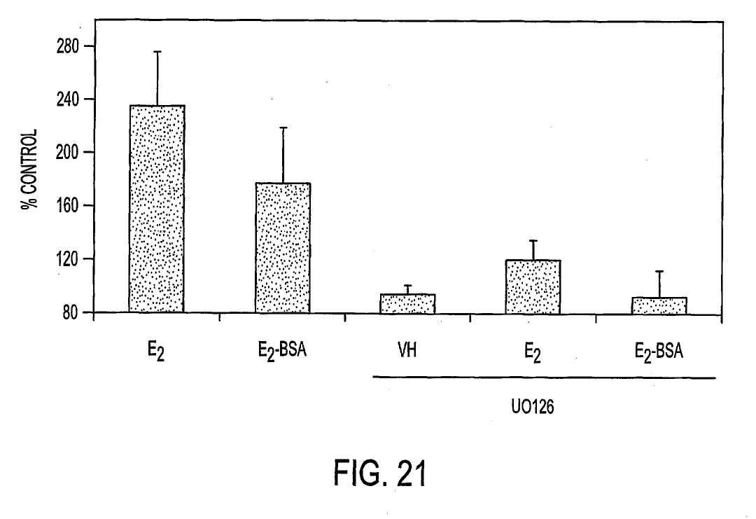

Cells were maintained in estrogen-free conditions 48 h before the experiment. In selected studies, cells were pre-incubated 90 min with U0126 (25 μM), a selective inhibitor of MEK1 and MEK2 (101) or for 2 h with anti-LBD Ab-2 (10 μg/ml) before treatment with estrogens. Protein samples were separated by SDS-PAGE and then transferred to a nitrocellulose membrane for immunodetection with anti-phospho-p44/p42 MAP kinase (Thr202/Tyr204) polyclonal antibody (New England Biolabs, Beverly, Mass., USA), using the Pierce Western blotting system. Akt activity was measured by Western blot using the Akt kinase assay kit (Cell Signaling Technology, Beverly, Mass., USA). After growth in estrogen-free conditions, cells were pre-incubated with anti-LBD Ab-2 (10 μg/ml), anti-PI(3) kinase inhibitor LY294002 (10 μM) (102) or ICI 182,780 (1 μM) (Astra Zeneca, Newark, Del., USA), followed by treatment with 10 nM E₂β or 0.5 μM E₂β-BSA for 20 min. Lysates were incubated overnight with anti-Akt kinase antibody. Thereafter, immunoprecipitates were processed for assay of Akt kinase activity according to recommendations of the manufacturer. Akt activity was assessed by densitometric analysis of phosphorylated GSK-3 using the public domain NIH Image program. FIG. 21 is a graph showing Inhibition of MCF-7 human breast cancer cell growth by the MEK 1/2 inhibitor U0126.

Membrane Labeling with Estradiol and Flow Cytometry

Single cell suspensions of MCF-7 cells from estrogen-free cultures were obtained using Versene (GIBCO BRL, Rockville, Md., USA). Cells were incubated at 4° C. for 10 min with 1 μM DCC-treated, fluorescein isothiocyanate (FITC)-labeled BSA with covalently-attached 17β-estradiol-hemisuccinate (E₂β-BSA-FITC) (52). For competition studies, cells were incubated 5 min with 100 nM E₂β, 1 μM E₂β-BSA, 1 μM ICI 182,780, 100 nM progesterone or 10 μg/ml ER antibody. A sample was analyzed by microscopy, and the remainder was used for flow cytometry using a FACScan with Cell Quest software (Beckton Dickinson, Franklin Lakes, N.J., USA). To facilitate nuclear staining, some cells were permeabilized with 0.1% Triton X-100 (61).

Human Tumor Xenografts in Nude Mice

MCF-7 cells were inoculated subcutaneously at 5×10⁷ cells/animal in the mid-back region of 3-mo-old female athymic mice (Charles River, Wilmington, Mass., USA) primed with E₂β in a biodegradable binder as before (91). Treatment was initiated when tumors grew to >30 mm³. Animals were randomized by weight and tumor size at the start of the experiment, with 6-8 animals included in each treatment group. Antibody and control solutions were administered by intraperitoneal injection.

Anti-ER LBD Ab2 was given at a dose of 3.5 mg/kg in 6 doses at 4-day intervals (over 26 days). Control injections with mouse IgG, (Pharmingen, San Diego, Calif., USA) were given on an identical treatment protocol.

### Example 2

### Plasma Membrane ER in Human Breast Cancer Cells

To assess the occurrence of membrane-associated estrogen receptor forms, we performed subcellular fractionation experiments in MCF-7 cells maintained in estrogen-free media. Cells were homogenized under controlled conditions and subcellular fractions were purified by established methods (48,71). Specific binding of [³H]-estradiol and immunoassay of ERα and 5′-nucleotidase, a plasma membrane marker enzyme, was then quantitated in subcellular fractions, including nuclear, microsomal, mitochondria-lysosomal, cytosol and plasma membrane fractions. The results show that estradiol binding activity, as well as ER-α and 5′-nucleotidase, show enrichment in particulate cell fractions including plasma membranes. About 20% of estradiol binding activity co-purifies with plasma membrane fractions from breast cancer cells.

In addition to nuclear binding of estradiol, estrogen-binding activity also shows significant co-localization with the plasma membrane marker enzyme, 5′-nucleotidase. In three experiments, enzyme-linked immunoassay of estrogen receptor (74,75) in particle-free extracts of whole homogenate and plasma membrane revealed that 15% of homogenate ER localized to plasma membranes. These experimental results are consistent with prior reports on the enrichment of estrogen-binding proteins in plasma membrane fractions from other estrogen-responsive tissues (see Table 1). Moreover, in the latter studies, estrogen-binding activity was solubilized from the membrane fraction and further resolved on 5-20% (w/v) sucrose density gradients with either 0.01 M or 0.4 M KCl. In low-salt gradients, macromolecule-bound estradiol sedimented at predominantly 7.4S, while in high-salt gradients, estrogen-binding activity occurred at both 3.6S and 4.9S (48). These results indicate that the estrogen-binding molecule in plasma membranes retains biologic stability during its purification to 3,000-4,000-fold (48) and provides evidence that our plan for isolation of membrane ER from breast cancer cells is feasible.

Enrichment of High-affinity Binding-sites with Specificity for E₂β in Breast Cancer Cell Plasma Membranes

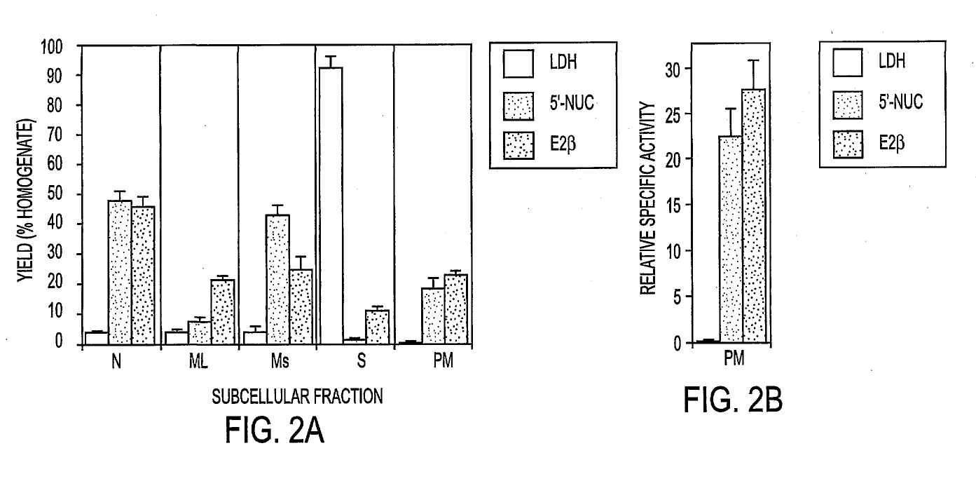

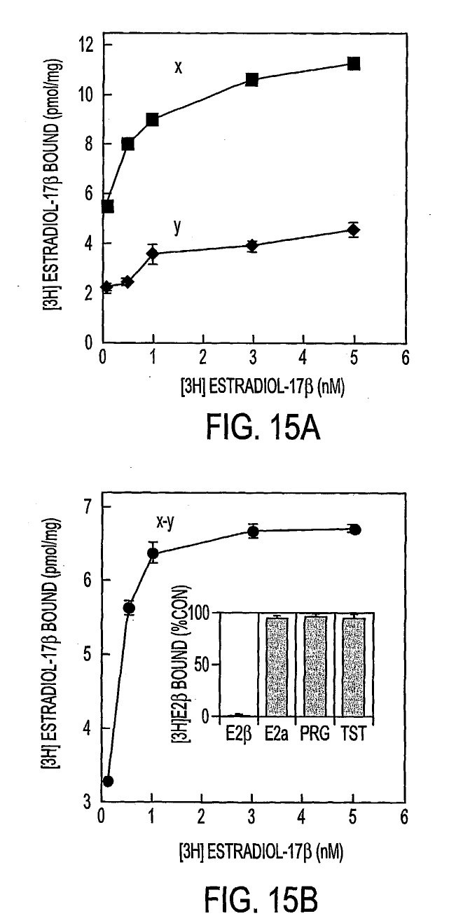

To confirm earlier reports of membrane binding-sites for E₂β we measured specific [³H]E₂β binding in subcellular fractions of MCF-7 cells after controlled cell homogenization and fractionation (47,48). With recovery of more than 97% of total E₂β binding found in homogenates of MCF-7 cells, specific [³H]E₂β binding was distributed among crude nuclear, microsomal, mitochondria-lysosome and cytosol fractions (FIG. 2a). After purification of plasma membranes from the crude nuclear fraction by use of discontinuous-sucrose density gradient centrifugation, the PM fraction showed enhanced activity of 5′-nucleotidase, a plasma membrane marker enzyme, to about 23-times that of homogenate (FIG. 2a, b). Specific [³H]E₂β binding in plasma membranes was enriched to 28-times homogenate activity and represented 22% of homogenate binding. This data shows that specific E₂β binding co-purifies with a plasma membrane marker protein in membrane fractions from breast cancer cells. LDH activity, highly enriched in cytosol, is not significantly detected in PM (FIG. 2 a, b). In addition, cell DNA recovery was 94±3% of homogenate levels in nuclear fractions, and no DNA was detected in PM fractions. Binding of [³H]E₂β by PM fractions from MCF-7 cells was analyzed further in equilibrium binding studies (FIG. 15). Samples of PM were exposed to [³H]E₂β concentrations ranging from 1×10⁻¹⁰ M to 5×10⁻⁹ M. As shown in FIG. 15a, all samples with [³H]E₂β alone retain greater amounts of hormone than paired samples in which [³H]E₂β was present together with a 100-fold molar excess of unlabeled hormone. The difference between the two curves, representing specific binding of E₂β, is plotted in FIG. 15b. It is evident that binding of hormone by PM is saturable, and Scatchard analyses of specific [³H]E₂β binding (cf. 48) show that the dissociation constant for the binding process is 3.6×10⁻¹⁰ M. Total binding sites in PM at saturation correspond to approximately 6.7 pmol E₂β per mg membrane protein. Further, ligand specificity of [³H]E₂β binding to PM was established by effective suppression by a 100-fold molar excess of unlabeled E₂β (FIG. 15b, inset). In contrast, [³H]E2β binding by PM was essentially uninfluenced by these levels of estradiol-17α, progesterone or testosterone.

Identification of Estrogen Receptor forms in Subcellular Fractions after Gel Electrophoresis

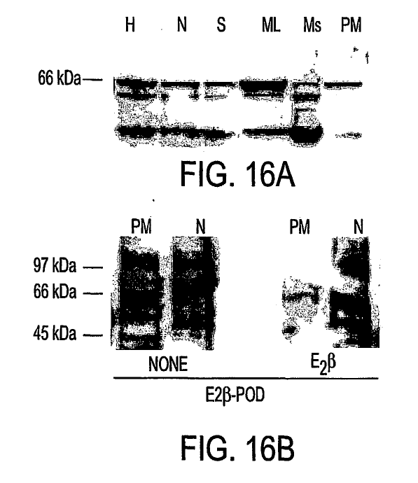

To characterize putative estrogen receptor forms associated with PM fractions, samples were subjected to Western blot analysis, and blots were probed either with anti-ER antibody Ab2 or with E₂β-POD (103). PM purified from MCF-7 cells show significant enrichment of a primary 67-kDa protein that reacts strongly with antibody Ab 2 to LBD of nuclear ER (FIG. 16a). Similarly, breast cell nuclear fractions are enriched with this protein reactive with ER antibody (FIG. 16a). The 67-kDa band also shows evidence of specific labeling with E₂β-POD (FIG. 16b). A secondary band at 46-kDa and minor bands at 62-kDa and 97-kDa were detected in PM and other cell fractions by use of Western blot (FIG. 16a) and ligand-blotting (FIG. 16b).

Identification of Estrogen Receptor Forms in Association with Caveola-related Membrane Domains

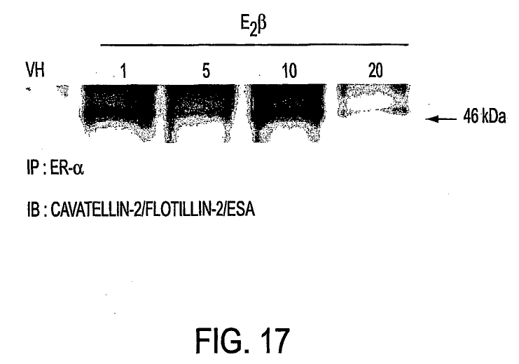

Caveolae and caveola-related domains are liquid-ordered regions of plasma membrane that are enriched in molecules that play important roles in intracellular signal transduction (Smart et al., Mol. Cell. Biol. 19: 7289, 1999). These molecules include receptor tyrosine kinases (HER-1, HER-2), components of the ras-MAPK pathways, PKC's, G-protein coupled receptors, etc. Consequently, caveolae and caveola-related domains function as critical preassembled signaling complexes for cross-communication between distinct signaling components. Here, we find that membrane-associated estrogen receptor forms in MCF-7 cells interact directly with cavatellin-2/flotillin-2, an integral membrane protein found in caveola-related domains (Volonte et al., J. Biol. Chem. 274: 12702, 1999) (see FIG. 17). Thus, the membrane-associated estrogen receptor appears to occur in association with a plasma membrane subcompartment that plays a crucial role in intracellular signal transduction (FIG. 17).

### Example 3

### Interaction of Membrane-associated Estrogen Receptor with Immobilized Estradiol and Alteration of Membrane-associated ER in Human Breast Cancer Cells with HER-2 Overexpression

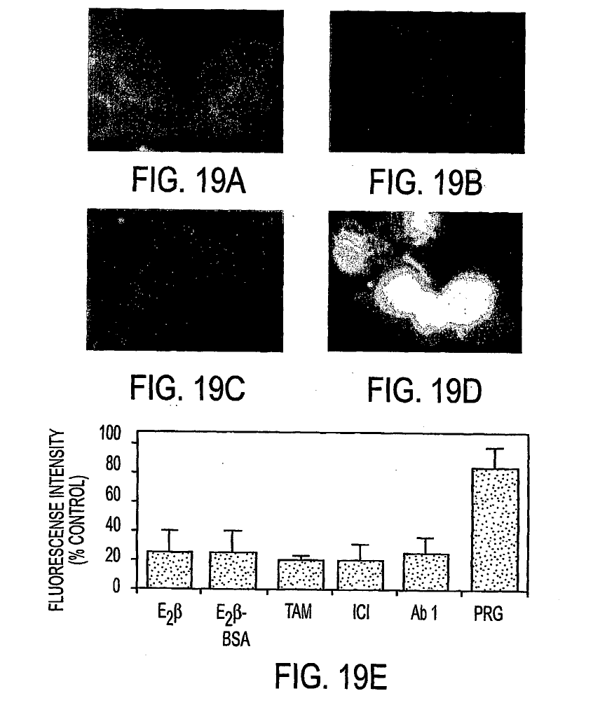

Several studies support the importance of a plasma membrane-associated form of estrogen receptor in mediating biologic actions of estrogen (46,47,56,61). As an alternate approach to assess the density of membrane-associated ER in intact human breast cancer cells, a fluorescent estradiol-bovine serum albumin conjugate (E₂β-BSA-FITC) was used for membrane labeling in vitro and then analyzed by microscopy and fluorescence-activated cell sorting by flow cytometry. Since interaction of E₂β-BSA with plasma membrane binding-sites may be required for intracellular signaling (52, 60, 61, 94, 106), we evaluated binding of fluorescein-labeled E₂β-BSA (E₂β-BSA-FITC) in MCF-7 cells. DCC-treated E₂β-BSA-FITC binds at the surface of 77% of MCF-7 cells (FIG. 19a), while only minimal background fluorescence is found among cells incubated with control ligand, BSA-FITC (FIG. 19b). On flow cytometric analysis (FIG. 19e), this macromolecular complex shows evidence of ligand specificity, with significant reduction (P<0.01) of E₂β-BSA-FITC binding by competition with equimolar amounts of free E₂β, E₂β-BSA, tamoxifen or ICI 182, 780, while the related steroid congener, progesterone, is not effective. Surface binding of E₂β-BSA-FITC is also significantly diminished by competition with antibody to the LBD of nuclear ER, providing evidence of some immunologic identity of the membrane site with nuclear ER (FIG. 19 c, e). As expected, after permeabilization of cells by disruption of plasma membrane with detergent, intense labeling of ER in cell nuclei is found and occurs in 96% of breast cancer cells (FIG. 19d).

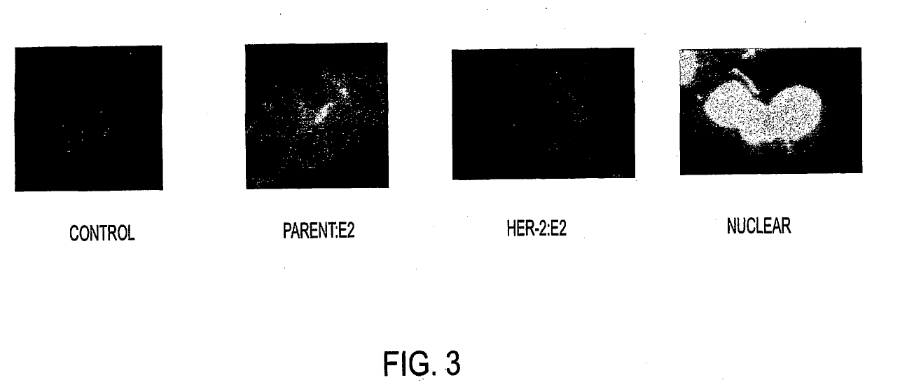

MCF-7 human breast cancer cells with (MCF-7 HER-2) or without (MCF-7 PAR) HER-2 receptor overexpression (3) were treated with 1 μM E₂β-BSA-FITC, a membrane-impermeant compound with relatively high binding affinity for ER (52,61). Labeling of membrane ER was found in both breast cancer cell lines (see FIG. 3), with 78% of MCF-7 PAR cells and 44% of MCF-7 HER-2 cells showing cell surface ER labeling (see TABLE 2 below). In MCF-7 PAR cells, membrane-associated binding of E₂β-BSA-FITC was competitively reduced by free estradiol, tamoxifen, ICI 182,780, excess E₂β-BSA, and by 1 μg/ml monoclonal antibody Ab 1 to the ligand binding domain of ER-α, but not by progesterone (see also, FIG. 19). To estimate total cell ER binding, breast cells were permeabilized with 0.05% Triton X-100, and 98% of MCF-7 PAR cells and 79% of MCF-7 HER-2 cells showed uptake of the estradiol conjugate. Of total cell ER binding based on measures of fluorescence intensity, membrane ER was estimated to be 3% of that found in MCF-7 PAR cells but was increased to 12% of that detected in MCF-7 HER-2 cells (see TABLE 2).

TABLE 2
Membrane labeling of MCF-7 breast cancer cells
with fluorescent estradiol conjugate*

| | E₂-BSA-FITC- | Plasma |
|---|---|---|
| Cell Group | Labeled Cells | Membrane Labeling |
| MCF-7 PAR | 78 + 12% (3) | 03 + 1% (3) |
| MCF-7 HER-2 | 44 + 7% (3) | 12 + 2% (3)† |
| *Fluorescent estradiol-BSA conjugate (E₂-BSA-FITC), a membrane-impermeant compound with high binding affinity for ER (52, 61), was DCC-treated and then used for membrane labeling of ER and analyzed by fluorescence-activated cell sorting. MCF-7 cells with (MCF-7 HER-2) or without (MCF-7 PAR) HER-2 overexpression were treated with 1 μM E₂β-BSA-FITC. Cell surface label was found in 78% of MCF-7 PAR cells and 44% of MCF-7 HER-2 cells in 3 experiments. To estimate | | |
| # total cell ER binding, cells were permeabilized with 0.05% Triton X-100, and 98% of MCF7 PAR cells and 79% of MCF-7 HER-2 cells then bound E₂-BSA-FITC. | | |

†P < 0.01 (student's t-test).

### Example 4

### Membrane ER Stimulates Proliferation of Breast Cancer Cells

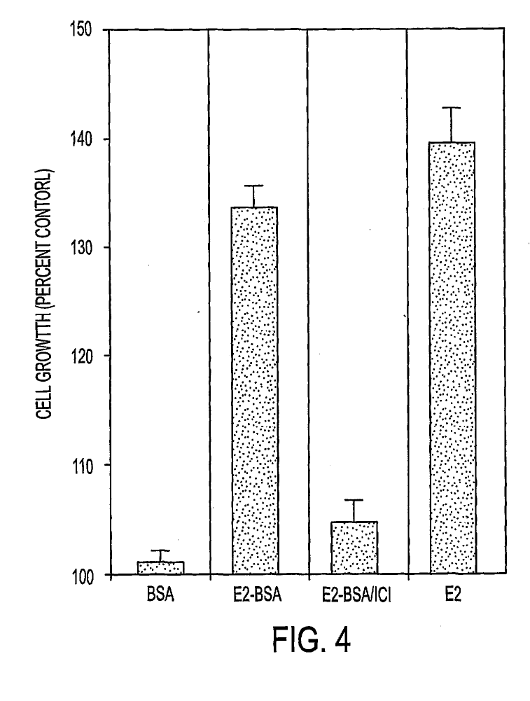

Some recent studies provide-evidence that the proliferative response to estrogen is committed within less than 1 minute of exposure to estrogen and appears to be evoked by the activation of only a small fraction (≦5%) of estrogen receptors (72,73). To test the proliferative response of breast cells to brief treatment with membrane-impermeable (61) estradiol-BSA conjugate (E₂β-BSA) and, thereby, assess the potential role of membrane ER in cell growth, we treated MCF-7 cells with 0.5 μM E₂β-BSA for only 10 min. Then, cells were rinsed and cultivated in estrogen-free media for an additional 48 h. The results show that E₂β-BSA, but not BSA, stimulates cell growth [see FIG. 4]. Moreover, the proliferative effect of E₂β-BSA is blocked by treatment of cells with the pure antiestrogen, ICI 182,780 [FIG. 4]. These data are consistent with earlier reports on stimulation of thymidine incorporation into DNA of ER-positive cells treated with E₂β-BSA (61).

FIG. 4 shows the proliferative response of MCF-7 breast cancer cells to short-term treatment with membrane-impermeable estradiol-bovine serum albumin conjugate (E₂-BSA). We treated MCF-7 cells with 200 nM E₂-BSA (E2-BSA), 200 nM BSA (BSA), 10 nM ICI 182,780 with 200 nM E₂-BSA or 20 nM estradiol for only 10 minutes. Then, cells were rinsed 3 times, cultivated in estrogen-free media for an additional 48 hrs and counted. The results are expressed as percent control (vehicle alone) and show that E₂-BSA and free estradiol, but not BSA, stimulate cell growth (P<0.001, t-test). Moreover, the proliferative effect of E₂β-BSA is blocked by treatment of cells with the pure antiestrogen, ICI 182,780.

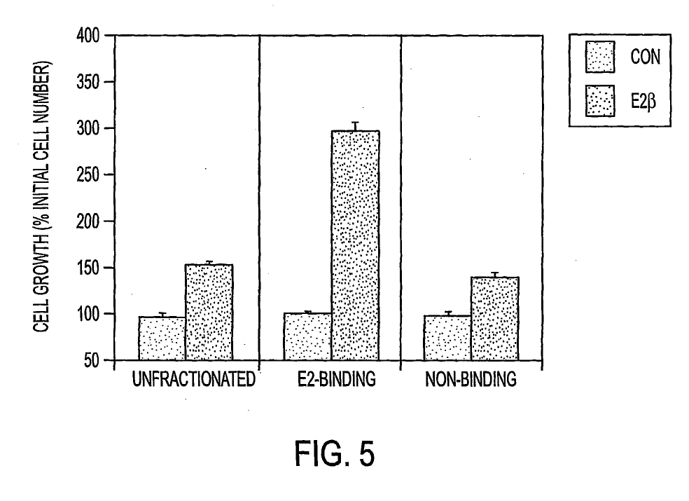

In parallel studies, we selected MCF-7 breast cancer cells with a capacity for binding immobilized estradiol using methods as before (46,47). FIG. 5 shows that estrogen elicits preferential growth of human breast cancer cells selected for expression of membrane estrogen receptor. To assess the importance of membrane ER in estrogen-induced cell growth, breast cancer cells were fractionated in vitro on the basis of their capacity to bind or not bind with 17β-estradiol-17-hemisuccinyl-albumin covalently bound to an inert support (46,47). Isolated MCF-7 breast cancer cells were cultivated in estrogen-free media for 72 hrs and then incubated for 30 min at 22° C. with immobilized estradiol at a prevailing concentration of approximately 0.5 nM as described before (47). These conditions were shown before to permit selection of cells with high affinity interactions with estradiol at the cell surface (46,47). No significant binding of cells was observed when inert supports coupled only to albumin were used, indicating specificity of cell binding to immobilized estradiol. Breast cancer cells bound to immobilized estrogen (E2-binding) were dislodged from the fibers in the presence of excess E2β and recovered intact by centrifugation (47). Corresponding cells which had not become bound to immobilized estradiol (non-binding), as well as cells not selected for binding to immobilized estrogen (unfractionated) were processed and recovered under parallel conditions. All cell groups were than cultivated for 3 days in estrogen-free media, followed by treatment with or without 2 nM estradiol-17β for 72 hrs. Cell numbers in all groups were quantitated and expressed relative to the initial cell number at the start of the treatments. The increment in estrogen-induced cell growth in E2-binding cells was significantly greater than that found in unfractionated and non-binding cell populations (P<0.001, t-test, 3 experiments).

### Example 5

### ER Associates Directly with HER-2 Receptor

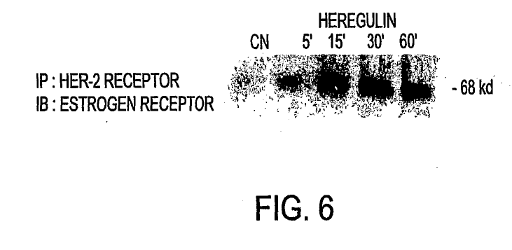

To further evaluate prior reports of cross-communication between ER and HER-2 receptors (3,24,29,54), MCF-7 PAR cells were treated in vitro with heregulin, a ligand for activation of HER-2/HER-3 receptor heterodimers (3). FIG. 6 shows that the activation of HER-2 growth factor receptor promotes physical association of HER-2 receptor with estrogen receptor (ER). MCF-7 breast cancer cells were treated in vitro for 5-60 minutes with 10 nM heregulin, a ligand known to activate HER-2/HER-3 receptors (3). Lysates were prepared and processed as described before (3). Samples were immunoprecipitated with anti-HER-2 antibody (IP: HER-2 receptor) prior to electrophoresis and Western blotting with anti-ER antibody H222 (IB: estrogen receptor). Estrogen receptor normally occurs as a 65- to 70-kd protein (3). The experiment shown here is representative of results from 4 other experiments. In independent experiments in which samples were immunoprecipitated with anti-ER antibody prior to electrophoresis and Western blotting with anti-HER-2 antibody, a similar association between ER and HER-2 receptors was found.

### Example 6

### Antisense Oligonucleotides to Intracellular ER Suppresses Membrane ER and Tumor Growth

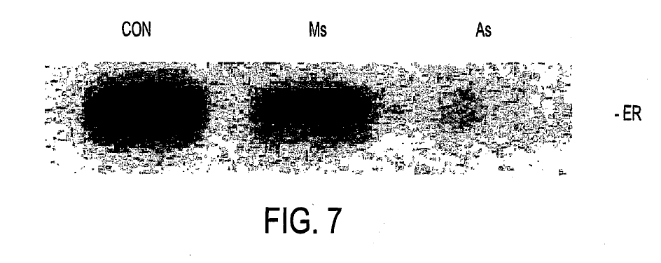

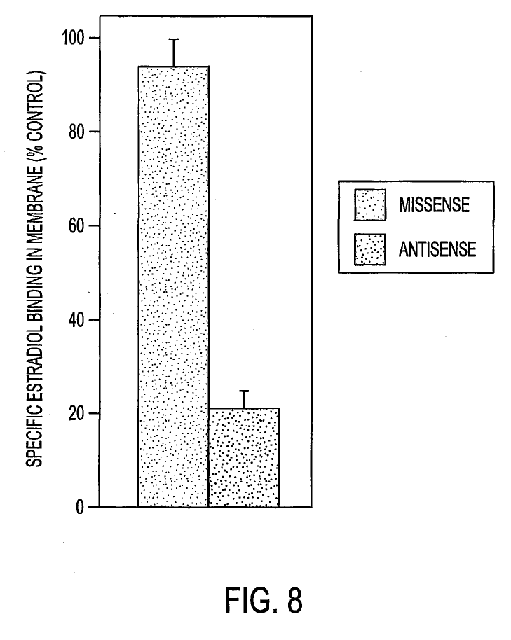

In view of increasing evidence for the existence and activity of a membrane from of ER (see Table 1), we devised an experiment to further evaluate the identity of membrane ER. MCF-7 breast cancer cells were treated with ER antisense oligonucleotide for suppression of nuclear ERα expression (84,85). As determined by use of Western blot techniques, suppression of the expression of ER protein is found after antisense treatment of MCF-7 cells for 48 hours (see FIG. 7). Moreover, on controlled homogenization and fractionation of MCF-7 cells treated with ER antisense, plasma membranes show significant reduction of membrane ER receptor levels (see FIG. 8).

FIG. 8 shows that antisense oligonucleotides to intracellular estrogen receptor reduce expression of membrane-associated receptors with specific high-affinity binding for estrogen. The antisense phosphorothioate oligonucleotide was synthesized as 5′-GGGTCATGGTCATGG-3′ (SEQ ID NO: 1), and a missense control was used for comparison. Specific estradiol-17β binding to plasma membrane fractions was done by established methods.

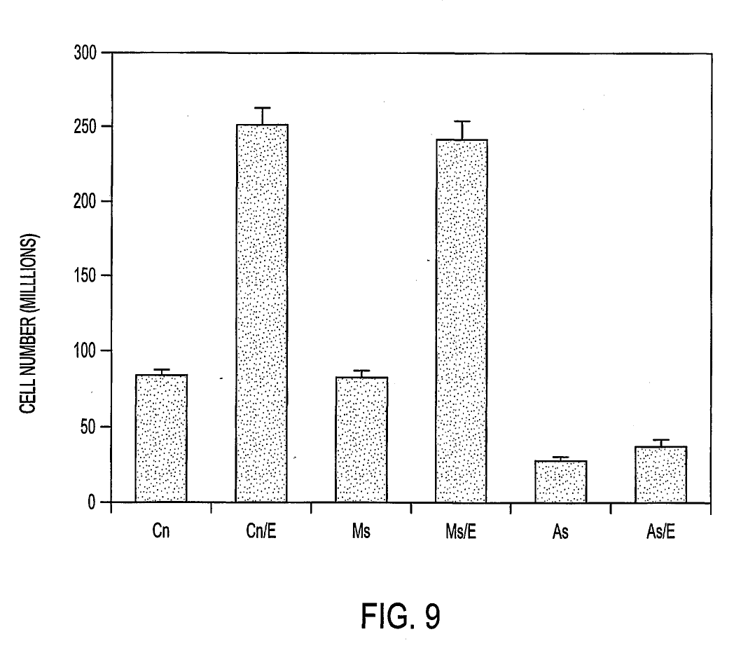

In addition, MCF-7 breast cancer cells display marked growth inhibition following ER antisense treatment (see FIG. 9). These results provide evidence consistent with the finding that membrane-associated estrogen receptors in breast cancer cells may derive, in part, from the same transcripts that yield the intracellular forms of ER.

### Example 7

### Inhibition of Estrogen-stimulated MAP Kinase Blocks Growth of Breast Cancer Cells

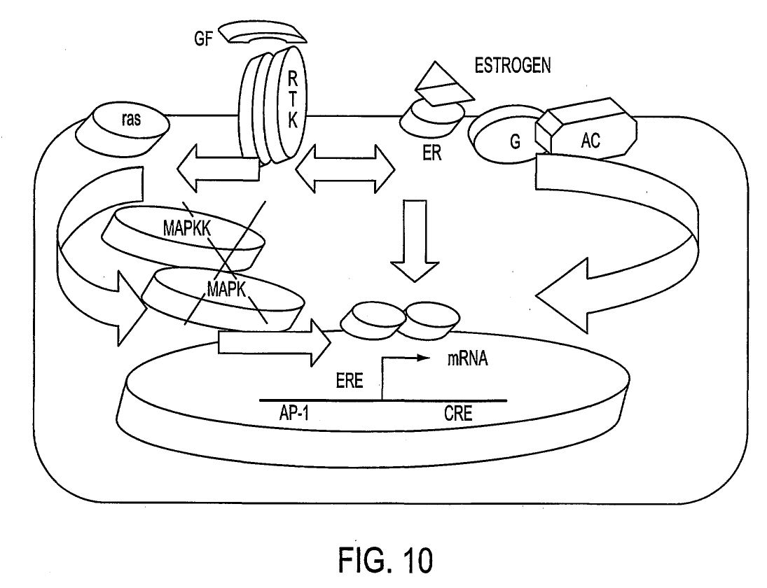

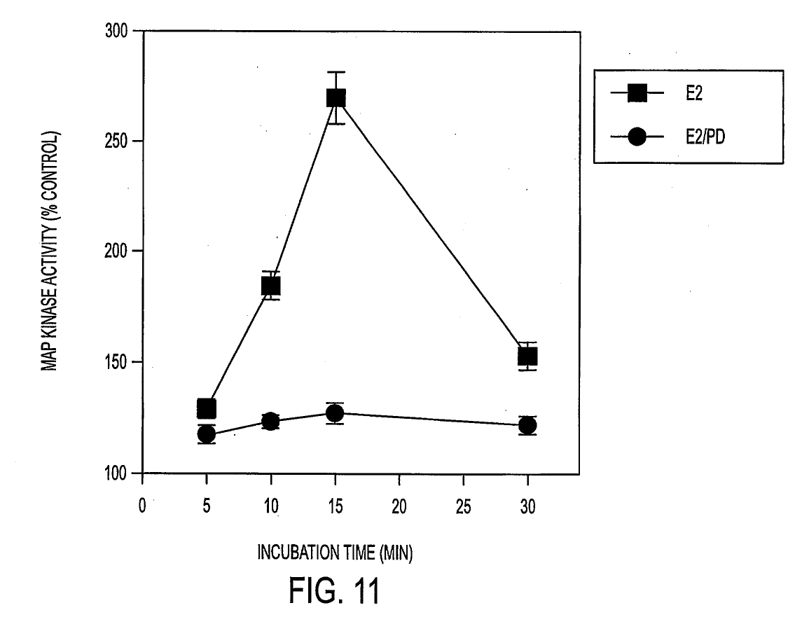

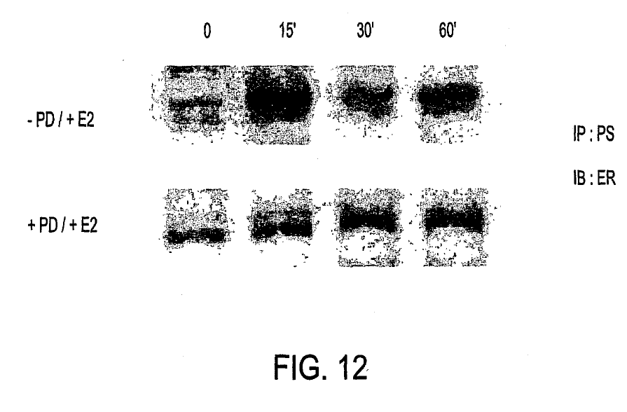

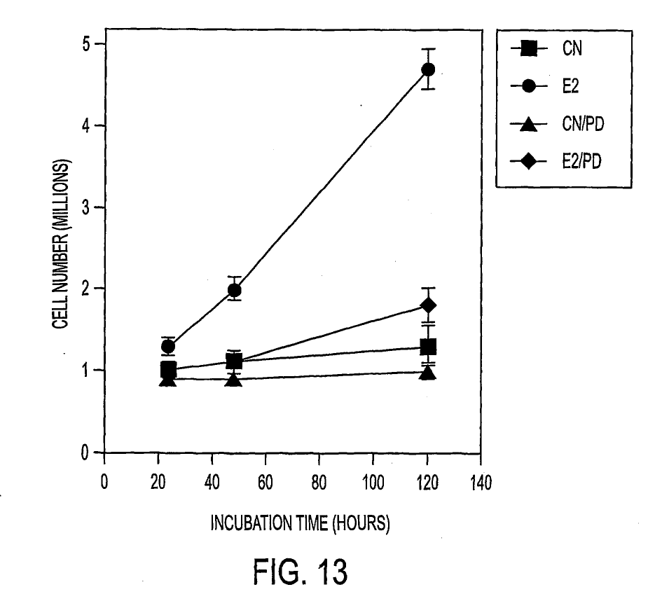

In view of prior studies suggesting potential interactions of ER with activation of mitogen-activated protein kinase (MAP kinase) signaling pathways (see Table 1 and FIG. 10), we investigated the activity of PD 98059, a specific inhibitor of MAPKK (MEK) that suppresses MAP kinase activation. In MCF-7 cells, estradiol promotes enhanced MAP kinase activity within 10-15 minutes in vitro (see FIG. 11), but this activation of MAP kinase is blocked by pre-treatment with PD 98059. Estrogen treatment leads, in turn, to increased serine phosphorylation of the estrogen receptor at 15 minutes, and this estrogen effect is also suppressed by prior exposure of cells to PD 98059 (see FIG. 12). This blockade of estrogen-induced MAP kinase activation by PD 980959 results in the suppression of estrogen-induced growth of MCF-7 breast cancer cells (see FIG. 13).

### Example 8

### Monoclonal Antibodies to Intracellular ER Block Growth-stimulatory Action of Membrane ER

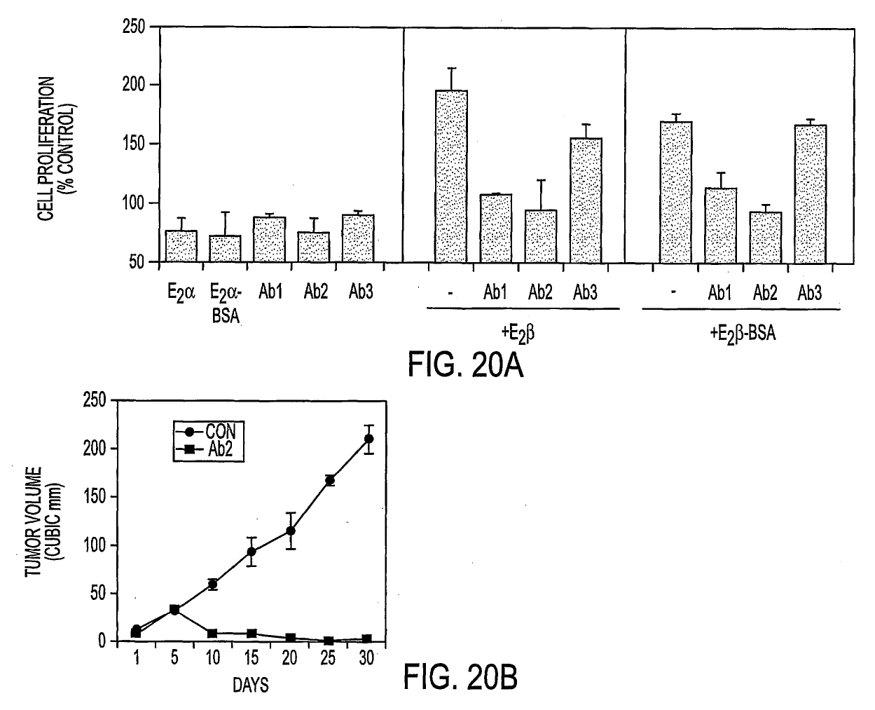

On the assumption that ligand-binding domains of ER may be accessible for interaction with specific antibodies at the surface membrane, monoclonal antibodies to different functional domains of intracellular estrogen receptor were tested for potential growth inhibitory activity in cultures of human MCF-7 breast cancer cells (see FIG. 20). As shown in FIG. 14, Ab 1 recognizes functional domains E (ligand-binding domain) and F in estrogen receptor (domains E and F localize approximately to amino acids 495-595). In addition, another monoclonal antibody designated Ab 2 recognizes functional domains D, E and F (domains D, E and F localize approximately to amino acids 302-595). Ab 3 is reactive with domains D and E (domains D and E localize approximately to amino acids 280-335)(FIG. 14).

Inhibition of Cell Growth in vitro by Antibody to Ligand-binding Domain of ER-α

Since antibodies to cell surface growth factor receptors are sometimes effective in blocking tumor cell growth (105), the antiproliferative activity of antibodies to ER-α was evaluated using MCF-7 cells in vitro. The estrogen-dependent MCF-7 cells show enhanced proliferation after treatment with E₂β, but not E₂α (FIG. 20a). However, prior exposure to LBD Ab1 or LBD Ab2 elicits a significant reduction (P<0.05) in the growth response to E₂β (FIG. 20a).

Since some recent studies provide evidence that the proliferative response to E₂β is committed within 1 min and is evoked by activation of only a small fraction (<5%) of ER (73), we assessed the growth of breast cells after brief treatment with E₂β-BSA. MCF-7 cells were treated with 0.5 μM E₂β-BSA for only 10 min. Then, cells were rinsed and cultivated in estrogen-free media for an additional 72 h. The results show that E₂β-BSA (P <0,001), but not control E₂α-BSA, stimulates cell growth (FIG. 20a). Moreover, the proliferative effect of E₂β-BSA is blocked by treatment of cells with ICI 182,780, a pure antiestrogen (P <0.001), or by prior exposure to anti-ER Ab1 (P<0.05) or Ab2 (P<0.001) (FIG. 20a).

Rapid Effects of E₂β and E₂β-BSA on Activation of MAPK and Akt Kinase in Breast Cancer Cells

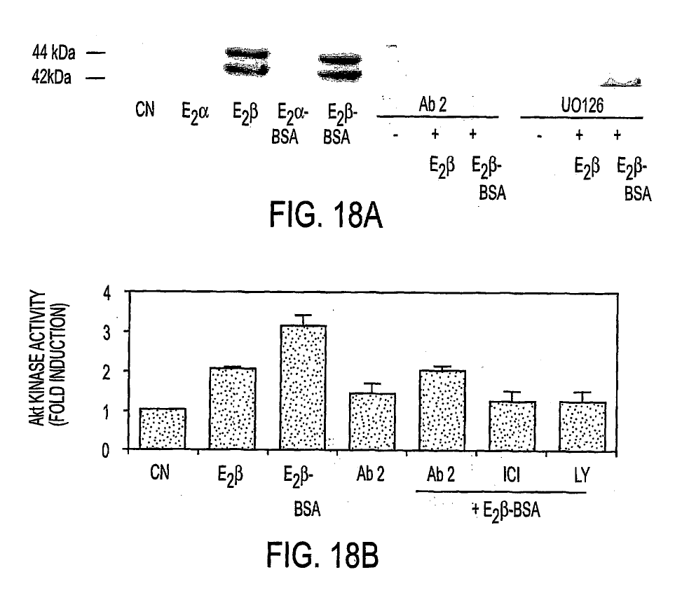

Post-receptor signal transduction events, such as stimulation of MAPK, extracellular signal-regulated kinase ERK-1 (p44) and ERK-2 (p42) (43, 104), may contribute to proliferative effects of E₂β in breast cells. Thus, we assessed estrogen-induced phosphorylation of MAPK in MCF-7 cells in vitro. E₂β, but not 17α-estradiol (E₂α), promotes phosphorylation of MAPK isoforms, with effects evident within 2 min (FIG. 18a). To test whether activation of MAPK by E₂β may be mediated by binding of estrogen to membrane-associated receptors, MCF-7 cells were treated with E₂13 linked to BSA, a macromolecular complex considered to be membrane-impermeant (52, 60, 61, 94). Using E₂β-BSA, but not control E₂α-BSA, phosphorylation of MAPK isoforms is again evident within 2 min of steroid administration. Incubation of cells with antibody against LBD of ER (Ab2) inhibited MAP kinase phosphorylation induced by E₂β or E₂β-BSA. Similarly, we assessed signaling via the phosphatidylinositol-3 kinase (PI3K)/Akt pathway after treatment of MCF-7 cells with E₂β or E₂β-BSA. Both ligands induced significant activation of Akt kinase (FIG. 18b), and inhibition of estrogen-induced effects occurred when cells were preincubated with ER antibody (Ab2), pure antiestrogen (ICI 182,780) or the PI3K inhibitor, LY 294002.

### Example 9

### Inhibition of Breast Tumorigenesis in vivo by Antibody to Ligand-binding Domain of ER-α

The antitumor activity of antibodies to ER-α was evaluated further using MCF-7 tumors in vivo. MCF-7 cells were grown as subcutaneous xenografts in female athymic mice primed with E₂β to promote growth of these estrogen-dependent cells (3). Antibody or control treatments were initiated when tumors grew to >30 mm³. Anti-ER Ab2 was administered in 6 doses over a 26-day period. The results show that antibody to ER, but not control immunoglobulin, elicits a significant suppression of tumorigenesis of human MCF-7 breast cancer xenografts in female nude mice treated concomitantly with E₂β (FIG. 20b).

### Example 10

### Assessing the Existence and Identity of Receptors for Estrogen in Membranes of Breast Cancer Cells

Many studies document the existence of membrane estrogen receptors, but the identity of these receptors remains elusive. These receptors may be known molecules (kinases, G proteins, ion channels) with unknown binding sites for estrogen, new isoforms of ERα or ERβ in membranes (38,61), classical ER complexed with other membrane-associated proteins or truly novel membrane proteins (65). This work can determine which interpretation is correct.

By use of ‘controlled subcellular fractionation’ methods (3,48,71), plasma membranes can be isolated from MCF-7 human breast cancer cells with and without overexpression of HER-2 receptor (FIG. 2). MCF-7 cells have been stably transfected with a vector containing the full-length cDNA of human HER-2 gene isolated from a primary breast cancer (3,15-17). These cells are termed MCF-7 HER-2. The vector for insertion of HER-2 into human cells contains full-length HER-2 gene ligated into a replication-defective retroviral expression vector, pLXSN (3,15-17). A vector devoid of HER-2 but with neomycin phosphotransferase gene for selection with G418 was packaged in an identical fashion and served as a control to infect MCF-7 cells (MCF-7 PAR). Pools of retrovirus-infected MCF-7 cells were selected for HER-2 receptor overexpression by fluorescence-activated cell sorting using monoclonal anti-HER-2 receptor antibody as described before (3) and screened further by subculture in the presence of G418. MCF-7 HER-2 cells with 2-5 copies of HER-2 gene per cell can be used in these studies. Cells can be routinely plated in RPMI medium 1640 (GIBCO) with 2 mM glutamine and 1% penicillin G-streptomycin-fungizone. For standard plating, medium with 10% heat-inactivated fetal bovine serum can be used. In membrane isolation experiments requiring estrogen-free conditions, medium without phenol red supplemented with 0.1% heat-inactivated, dextran-coated charcoal-treated FBS can be used for 72 h prior to starting the experiment (3). In some studies, cells can be pre-selected for those with or without membrane estrogen-binding activity and expanded independently in culture prior to harvesting for subcellular fractionation (47; see FIG. 4).

Following collection of subcellular fractions and plasma membrane in the presence of proteinase inhibitors (3), activity of ERα can be determined by two independent approaches, ligand binding with [³H]-estradiol-17β (3) and enzyme-linked immunoassay (ELISA) (74,75). Differences in the two approaches may be found if membrane ER originate from a transcript other than classical ERα. If this occurs, one skilled in the art can consider use of ligand affinity blotting techniques to characterize alternative estrogen-binding membrane molecules (FIG. 16)(60,61). Prior studies show that MCF-7 cells do not contain significant levels of ERβ (76). Verification of plasma membrane purity can be confirmed as before by use of enzyme markers (48,71) and ELISA assay of HER-2 (77).

On solubilization of candidate receptor molecules from membranes (see FIG. 2; 48,51,78), further purification can be achieved by an affinity chromatography approach. The method is described in detail by Greene at al.(79), and a similar approach by Puca et al. (80) for purification of ER from calf uterus was also successful. Subsequent molecular characterization of any isolates can be done using established techniques (48,78,79,81). We expect that recovered receptor can be available for preparation of monoclonal antireceptor antibodies by established methods (79) and for peptide sequencing (82,83). Synthetic oligonucleotide probes can be designed from peptide sequences of membrane ER and can then be used to isolate clones corresponding to ER from randomly primed cDNA libraries as described before (82). The availability of these molecular tools for cell transfection studies (82, 83) could rapidly advance our understanding of the role of membrane-associated ER in breast cancer.

One skilled in the art can also prepare plasma membranes for comparison from MCF-7 cells treated with ER antisense oligonucleotide (0.35 μM for 24 h) in order to suppress expression of ERα (84,85) and from COS-7 cells with and without transfected ERα and ERβ for additional control experiments (61,76). The approach disclosed above may provide definitive information on the identity of membrane ER molecules.

### Example 11

### Assessing the Role of Membrane Estrogen Receptors in Promoting Growth of Breast Cancers

This Example focuses on signal transduction by the estrogen receptor. It is presently unclear whether membrane-associated ER and nuclear ER perform different functions or provide redundant or even synergistic signaling. One skilled in the art can focus on interactions of membrane ER with HER-2 receptor and the activation of mitogen-activated protein kinase (MAP kinase), but effects on other signaling pathways, such as PI3K/Akt kinase, can also be considered. Current data from studies on a variety of target cells provide evidence that estradiol elicits rapid downstream effects to activate membrane G proteins (61,67), inositol phosphate production (61), adenylate cyclase activity (58,61), calcium (39,40,58) and HER-2 tyrosine kinase [see TABLE 1]. These signaling cascades appear to stimulate MAP kinase and promote the later activation of transcription, DNA synthesis and growth (61). However, in some human breast cancer cells, the activation of MAP kinase by estrogen may occur by a more defined signaling pathway involving calcium mobilization (40). Our studies can focus on estrogen signaling in MCF-7 cells with expression of ER and with or without overexpression of HER-2, as well as with ER-null cells and daughter cells stably transfected with cDNA for membrane ER and ERα using established procedures (3). Specific methods for assay of signaling pathway components can include:

Activation of HER-2 tyrosine kinase and association with ER: Human breast cancer cells can be tested for tyrosine phosphorylation of HER-2 receptor with Western blot using gel electrophoresis as before (3,15). In brief, tyrosine phosphorylation of HER-2 can be assessed using immunoprecipitation of cell lysate protein with agarose-conjugated anti-phosphotyrosine monoclonal antibody (Upstate), followed by immunoblotting with anti-HER-2 antibody (3). Physical interaction of HER-2 receptor with membrane ER, ERα and ERβ, following heregulin or estrogen stimulation, can be assessed further using methods described elsewhere (3) (FIG. 6).

MAP kinase activation: Activity of MAP kinase in response to estradiol in MCF-7 and control cells can be evaluated further in vitro using a specific antibody recognizing the active, phosphorylated forms of p44/p42 MAPK (Erk1/Erk 2) for Western immunoblot analyses as before (40). Western immunoblots of the same cell extracts from MCF-7 and control cells can also be done using an antibody that recognizes both nonphosphorylated forms of p44/p42 MAP kinase in order to assess whether treatment alters MAP kinase activity and/or expression (FIG. 11).

Calcium homeostasis: Intracellular calcium concentrations can be measured by using the ratiometric fluorescent indicator dye Fura 2-AM, a membrane-permeant acetoxymethyl ester form of Fura 2 (Molecular Probes). Confluent MCF-7 cell monolayers grown on coverglasses can be incubated with 10 μM Fura 2-AM using established protocols, with calculation of calcium concentration as before (40). As required, alternate measures of calcium flux can also be considered (39).

G protein activation: To assess activation of G proteins by membrane ER, binding of guanosine 5′-3-O-(thio)triphosphate (GTPγS) to Gαs or Gαq can be tested in membrane preparations as described before (40,61).

Adenylate cyclase activity: To assess activation of adenylate cyclase activity by membrane ER, cAMP generation can be measured by established procedures (40,41,61)

Inositol phosphate production: Generation of inositol phosphate (IP₃)can be assayed as before (40,61).

Ligands for testing in these experiments can include free estradiol-17β, membrane-impermeant estradiol-17β-BSA (61), BSA (control), estradiol-17α (stereospecific control), and progesterone (steroid control). To investigate potential inhibitory effects, ligands in some experiments can be used in combination with antibodies to membrane ER or to classical ER functional domains (H-222, Abbott; domain-specific Neomarkers antibodies) to determine the accessibility of membrane ER to modulation by cell surface-reactive agents; antibodies to HER-2 (Herceptin); or antiestrogens, including tamoxifen, the pure anti-estrogen, ICI 182,780, or raloxifene. The association of membrane ER signaling with transcriptional events and with cell growth (61) can be assessed further by established methods as below:

Transient Transfection of Breast Cancer Cells with ERE-CAT Reporter Gene Constructs:

For testing transcriptional activation by membrane ER, a reporter plasmid containing palindromic ERE and the chloramphenicol acetyltransferase (CAT) gene can be used in these studies (ERE-CAT; 3). Substitution of the basic reporter plasmid pBLCAT2 for pERE-BLCAT in selected experiments can provide a control for specificity of the DNA-binding site in the regulatory sequence of the reporter gene. The plasmid pCMV, which contains the β-galactosidase gene, can be used as an internal control for transfection efficiency (3). Cells can be transfected as before (3). Activity of test reagents can be assessed using transfected cells with or without ERE-CAT. Cells can be harvested 24 h later, and CAT protein can be quantitated in cell extracts using a non-radioactive enzyme-linked immuno-sorbant assay (5′-3′, Boulder, Col.) as before (3). CAT reporter activity can be normalized for protein in each sample. Depending on results of the latter studies and ongoing research in this area (38,76), one skilled in the art may also consider assessing the transactivation properties of membrane ER in the context of an AP1 site, using transfection of an AP1 reporter plasmid in cells expressing membrane ER.

Activation of Growth in Human Breast Cancer Cells:

MCF-7 cells can be cultivated in estrogen-free media for 72 h prior to the start of proliferation experiments. As noted above, ligands for testing can include free estradiol-17β, estradiol-17β-BSA (membrane-impermeant [61]), BSA (control), estradiol-17α (control), and progesterone (control), and growth can be quantitated by cell counts at the end of the experiment as before (3) (FIG. 20).

### Example 12

### Investigating Alternate Treatments to Prevent Breast Cancer Progression in Models of Breast Cancer

It is well-established that cross-talk between erb B/HER receptor pathways and ER may occur, and these interactions may be central to the failure of antiestrogen therapy in patients with HER2-overexpressing breast cancers (20-31). Erb B/HER-reactive peptides are known to modulate ER-dependent transcription in the absence of estrogen, in part by downstream pathways involving MAP kinase (86-88). Similarly, activation of HER-2 signaling by heregulin supports growth of estrogen-dependent MCF-7 cells in the absence of estrogen (3). Blockade of estrogen-induced growth of MCF-7 cells by tyrosine kinase inhibitors further attests to the importance of tyrosine kinases in estrogen action (89). Phosphorylation of ER itself on tyrosine and serine residues elicits functional changes in the interaction of ER with ERE (3,87,88). Thus, the transcriptional activity of ER may be modulated independent of estrogen by phosphorylation of ER through growth factor signaling pathways (3,86-89; see FIG. 1).

With the emergence of these novel modes of estrogen action (FIG. 1), one skilled in the art can evaluate new approaches to antiestrogen therapy that may offer patients fewer side-effects and greater antitumor efficacy. A number of important breast cell models can be available for use in these experiments. These include:

a) MCF-7 breast cancer cells with expression of ER-α, with and without overexpression of HER-2 or HER-1/EGFR (3)

b) ER-null breast cells and daughter cells stably transfected with cDNA for ER-α (3,61)

c) MCF-7 cells treated with ER antisense oligonucleotide for suppression of ER-α expression (84,85)

d) ER-null COS-7 and CHO cells; COS-7 and CHO daughter cells stably transfected with ER-α cDNA (3, 61)

e) COS-7 and CHO daughter cells stably transfected with cDNA for ER-α as well as cDNA for either HER-1, HER-2, HER-3 or HER-2/HER-3 together (90). ER-null COS-7 and CHO cells stably transfected with cDNA for either HER-1, HER-2, HER-3 or HER-2/HER-3 together can be available for these studies and can also be used to examine the report of direct interactions between estradiol and HER receptors (see 54)

f) ER-positive breast cancer cells selected for binding to immobilized estradiol (46,47; see FIG. 4)

Cell models with qualitative and quantitative differences in membrane ER-α and HER receptor expression (see above) can be tested for their differential growth sensitivities to antiestrogens, including tamoxifen, ICI 182,780 and raloxifene. Patient management decisions in the clinic may be greatly aided by a simple understanding of how estrogen and erb B/HER growth factor pathways function to promote growth of the cancer cell and how different levels of receptor expression affect the response to standard antiestrogen therapies.

Prior experiments have demonstrated that antibodies directed to ER may disrupt cellular actions triggered by a plasma membrane-associated receptor (50,61). In view of the success of antireceptor antibodies in the treatment of human breast cancers (3,91), one skilled in the art can assess the potential antitumor activity of new antibodies directed to membrane-associated estrogen receptors, alone and in combination with antibodies to HER-2 (Herceptin) and current antiestrogens, using methods as before (3). Alternate strategies could involve targeting the downstream signaling pathways that emanate from activation of the membrane-associated estrogen receptor. Development of new cell surface membrane-directed immunologic and biologic therapies may offer breast cancer patients targeted antitumor therapy with reduced systemic toxicity.

By attempting to isolate and purify the membrane-associated estrogen receptor from human breast cancer cells, this research challenges the conventional dogma that estrogen acts exclusively via a nuclear-localized receptor. The most exciting aspect of these studies is that they may offer a paradigm for a potentially large new class of drugs. Rational drug design strategies disclosed in this proposal may result in the development of new and previously unsuspected antitumor agents targeted to human breast cancer cells.

### REFERENCES

1.) Harris J., M. Lippman, U. Veronesi & W. Willeff (1992). Breast cancer. N. Engl. J. Med., 327: 473-451.

2.) Aaronson S. A. (1991). Growth factors and cancer. Science, 254: 1146-1152.

3.) Pietras, R. J., Arboleda, J., Wongvipat, N., Ramos, L., Parker, M. G., Sliwkowsli, M. X., and Slamon, D. J. (1995). HER-2 tyrosine kinase pathway targets estrogen receptor and promotes hormone-independent growth in human breast cancer cells. Oncogene, 10 : 2435-2446.

4.) Carpenter G. and S. Cohen (1979). Epidermal growth factor. Ann. Rev. Biochem., 48:193-208.

5.) Bishop J. M. (1983). Cellular oncogenes and retroviruses. Ann. Rev. Biochem., 52: 301-318.

6.) Lieberman T. A., H. R. Nusbaum, N. Razon, R. Kris, I. Lax, H. Soreq, N. Whittle, M. D. Waterfield, A. Ullrich and J. Schlessinger (1985). Amplification, enhanced expression and possible rearrangement of the epidermal growth factor receptor gene in primary human tumors of glial origin. Nature, 313:144-147.

7.) Dotzlaw H., T. Miller, J. Karvelas and L. C. Murphy (1990). Epidermal growth factor gene expression in human breast cancer biopsy samples : relationship to estrogen and progesterone receptor gene expression. Cancer Res., 50 : 4204-4212.

8.) Gullick W., J. Marsden, N. Whittle, B. Ward, L. Bobrow & M. Waterfield (1986). Expression of the epider-mal growth factor receptors on human cervical, ovarian and vulvar carcinomas. Cancer Res., 46: 285-293.

9.) Coussens L., T. C. Yang-Feng, Y. C. Liao, E. Chen, A. Gray, J. McGrath, P. H. Seeburg, T. A. Lieberman, J. Schlessinger et al.(1985). Tyrosine kinase receptor with extensive homology to EGF receptor shares chromosomal location with neu oncogene. Science, 230: 1132-1135.

10.) Schechter A. L., D. F. Stem, L. Vaidyanathan et al.(1985). The neu oncogene: an erb B related gene coding a 185,000-M tumor antigen). Nature, 312: 513-515.

11.) Semba K., N. Kamata, K. Toyoshima and T. Yamamoto (1985). A v-erbB-related proto-oncogene, c-erb B2, is distinct from the c-erbB-1 epidermal growth factor receptor gene and is amplified in a human salivary gland adenocarcinoma. Proc. Natl. Acad. Sci. USA, 82: 6479-6486.

12.) Kraus M. H., P. Fedi, V. Starks, R. Muraro and S. A. Aaronson (1993). Demonstration of ligand-dependent signaling by the erbB-3 tyrosine kinase and its constituitive activation in human breast tumor cells. Proc. Natl. Acad. Sci. USA, 90: 2900-2905.

13.) Plowman G. D., J. M. Culouscou, G. S. Whitney, J. M. Green, G. W. Carlton, L. Foy, M. G. Neubauer and M. Shoyab (1993). Ligand-specific activation of HER4/pl8oerbB4, a fourth mamber of the epidermal growth factor receptor family. Proc. Natl. Acad. Sci. USA, 90: 1746-1752.

14.) Culouscou J. M., G. D. Plowman, G. W. Carlton, J. M. Green & M. Shoyab (1993). Characterization of a breast cancer cell differentiation factor that specifically activates the HER4/p180erbB4 receptor. J. Biol. Chem., 268:18407-18416.

15.) Slamon D. J., G. M. Clark, S. G. Wong et al.(1987). Human breast cancer: Correlation of relapse and survival with amplification of the HER-2/neu oncogene. Science, 235: 177-181.

16.) Slamon D. J., W. Godolphin, L. A. Jones, J. A. Holt, S. G. Wong, D. E. Keith, W. J. Levin, S. G. Stuart, J. Udove, A. Ullrich and M. F. Press (1989). Studies of the HER-2/neu proto-oncogene in human breast and ovarian cancer. Science, 244 : 707-711.

17.) Slamon D., M. Press, W. Godolphin, L. Ramos, P. Haran, L. Shek, S. Stuart and A. Ullrich (1989). Studies of the HER-2/neu oncogene in human breast cancer. Cancer Cells, 7: 371-378.

18.) Joshi M., A. Lee, M. Loda, M. Camus, C. Pedersen, G. Heatley and K. Hughes (1996). Male breast cancer: An evaluation of prognostic factors contributing to a poorer outcome. Cancer 77: 490-498.

19.) Tiwari R., P. Borgen, G. Wong, C. Cordon-Cardo, and M. Osborne (1992). HER-2/neu amplification and overexpression in primary human breast cancer is associated with early metastasis. Anticancer Research 12: 419-425.

20.) Wright C., B. Angus, S. Nicholson et al. (1989). Expression of c-erbB-2 oncoprotein: a prognostic indicator in human breast cancer. Cancer Res., 49: 2087-2094.

21.) Nicholson S., C. Wright, J. R. C. Sainsbury, P. Halcrow, P. Kelly, B. Angus, J. R Farndon and A. L. Harris (1990). Epidermal growth factor receptor as a marker for poor prognosis in node-negative breast cancer patients : neu and tamoxifen failure. J. Steroid Biochem., 37: 811-818.

22.) Klijn J., P. Berns, P. Schmitz, J. Foekens (1992). The clinical significance of epidermal growth factor receptor in human breast cancer : review on 5232 patients. Endocr. Rev., 13: 3-15.

23.) Wright C., S. Nicholson, B. Angus, J. R. Sainsbury, J. Famdon, J. Cairns, A. L. Harris and C. H. Home (1992). Relationship between c-erbB-2 protein product expression and response to endocrine therapy in advanced breast cancer. Br. J. Cancer, 65: 118-124.

24.) Benz C., G. Scott, J. Sarup, R. Johnson, D. Tripathy, E. Coronado, H. Shepard and C. Osborne (1993). Estrogen-dependent, tamoxifen-resistent tumori-genic growth of MCF-7 cells transfected with HER2/neu. Breast Cancer Res. Treatment, 24: 85-92.

25.) Borg A., B. Baldetorp, M. Ferno, D. Killander, H. Olsson, S. Ryden & H. Sigurdsson (1994). ErbB2 amplification is associated with tamoxifen resistance in steroid-receptor positive breast cancer. Cancer Letters, 81:137-143.

26.) Leitzel K., Y. Teramoto, K. Konrad, V. Chinchilli, G. Volas, H. Grossberg, H. Harvey, L. Demers, and A. Lipton (1995). Elevated serum c-erbB-2 antigen levels and decreased response to hormone therapy of breast cancer. J. Clin. Oncol., 13: 1129-1135.

27.) Newby J., S. Johnston, I. Smith and M. Dowsett (1997). Expression of epidermal growth factor receptor and c-erbB2 during the development of tamoxifen resistance in human breast cancer. Clin. Cancer Res., 3: 1643-1651.

28.) De Placido S., C. Carlomagno, M. De Laurentiis and A. Bianco (1998). c-erbB2 expression predicts tamoxifen efficacy in breast cancer patients. Brst. Cancer Res. Trtmt., 52: 55-64.

29.) Nass, S., H. Hahm and N. Davidson (1998). Breast cancer biology blossoms in the clinic. Nature Medicine, 4: 761-762.

30.) Pegram, M., G. Pauletti and D. Slamon (1998). Her-2/neu as a predictive marker of response to breast cancer therapy. Brst. Cancer Res. Trtmt., 52: 65-77.

31.) Houston S., Plunkett T., Barnes D., Smith P., Rubens R., and Miles D (1999). Overexpression of c-erbB2 is an independent marker of resistance to endocrine therapy in advanced breast cancer. British Journal of Cancer, 79:1220-1226.

32.) Green S. and P. Chambon (1986). A superfamily of potentially oncogenic hormone receptors. Nature, 324: 615-618.

33.) Beug H. and T. Graf (1989). Cooperation between viral oncogenes in avian erythroid and myeloid leukaemia. Eur. J. Clin. Invest., 19: 491-501.

34.) Read L., D. Keith, D. Slamon and B. Katzenellenbogen (1990). Hormonal modulation of HER-2/neu protooncogene messenger ribonucleic acid and pl85 protein expression in human breast cancer cell lines. Cancer Res., 50: 3947-3955.

35.) Russell, K. and M. -C. Hung (1992). Transcriptional repression of the neu protooncogene by estrogen stimulated estrogen receptor. Cancer Res., 52: 6624-6632.

36.) Tang C. K., C. Perez, T. Grunt, C. Waibel, C. Cho and R. Lupu (1996). Involvement of heregulin-β2 in the acquisition of the hormone-independent phenotype of breast cancer cells. Cancer Research, 56: 3350-3358.

37.) Green, S. and P. Chambon (1988). Nuclear receptors enhance our understanding of transcription regulation. Trends Genet., 4: 309-314.

38.) Karin, M. (1998). New twists in gene regulation by glucocorticoid receptor: Is DNA binding dispensable? Cell, 93 : 487-490.

39.) Pietras, R. and Szego, C. (1975). Endometrial cell calcium and oestrogen action. Nature, 253: 357-359.

40.) Improta-Brears, T., A. Whorton, F. Codazzi, J. York, T. Meyer and D. McDonnell (1999). Estrogen-induced activation of mitogen-activated protein kinase requires mobilization of intracellular calcium. Proc. Natl. Acad. Sci. USA, 96: 4686-4691.

41.) Szego, C. and Davis, J. (1969). Adenosine 3′,5′-monophosphate in rat uterus : acute elevation by estrogen. Proc. Natl. Acad. Sci. USA, 58 :1711-1715.

42.) Aronica, S., Kraus, W. and Katzenellenbogen, B. (1994). Estrogen action via the cAMP signaling pathway: stimulation of adenylate cyclase and cAMP-regulated gene transcription. Proc. Natl. Acad. Sci. USA, 91:8517-8521.

43.) Migliaccio, A., Di Domenico, M., Castoria, G., de Falco, A., Bontempo, P., Nola, E. and Auricchio, F. (1996). Tyrosine kinase/p21ras/MAP-kinase pathway activation by estradiol-receptor complex in MCF-7 cells. EMBO Journal, 15:1292-300.

44.) Endoh, H., Sasaki, H., Maruyama, K., Takeyama, K., Waga, I., Shimizu, T., Kato, S. and Kawashima, H. (1997). Rapid activation of MAP kinase by estrogen in the bone cell line. Biochem. Biophys. Res. Commun., 235:99-102.

45.) Le Mellay, V., B. Grosse and M. Lieberherr (1997). Phospholipase C β and membrane action of calcitriol and estradiol. J. Biol. Chem., 272: 11902-11907.

46.) Pietras, R. and Szego C. (1977). Specific binding sites for oestrogen at the outer surfaces of isolated endometrial cells. Nature, 265:69-72.

47.) Pietras R. Szego C. (1979). Metabolic and proliferative responses to estrogen by hepatocytes selected for plasma membrane binding-sites specific for estradiol-17β. J. Cellular Physiology, 98:145-159.

48.) Pietras, R and C. Szego (1980). Partial purification and characterization of oestrogen receptors in subfractions of hepatocyte plasma membranes. Biochem. J., 191: 743-760.

49.) Nenci, I., Fabris, G., Marchetti, E. and Marzola, A. (1980). Cytochemical evidence for steroid binding sites in the plasma membrane of target cells. In Perspectives in Steroid Receptor Research (Ed. by F. Bresciani), Raven Press, New York: pp.61-69.

50.) Pietras, R., Szego, C. and Seeler, B. (1981). Immunologic inhibition of estrogen binding and action in preputial gland cells and their subcellular fractions. J. Steroid Biochem., 14: 679-691.

51.) Pietras R. J. and C. M. Szego (1984). Specific internalization of estrogen and binding to nuclear matrix in isolated uterine cells. Biochem. Biophys. Res. Commun., 123: 84-90.

52.) Berthois, Y., N. Pourreau-Schneider, P. Gandilhon, H. Mittre, N. Tubiana and P. Martin (1986). Estradiol membrane binding sites on human breast cancer cell lines. Use of a fluorescentn estradiol conjugate to demonstrate plasma membrane binding systems. J. Steroid Biochem., 25: 963-972.

53.) Lieberherr, M., Grosse, B., Kachkache, M and Balsan, S. (1993). Cell signaling and estrogens in female rat osteoblasts: a possible involvement of unconventional non-nuclear receptors. J. Bone Mineral Res., 8: 1365-1376.

54.) Matsuda, S., Y. Kadowaki, M. Ichino, T. Akiyama, K. Toyoshima and T. Yamamoto (1993). 17β-Estradiol mnimics ligand activity of the c-erb B2 protooncogene product. Proc. Natl. Acad. Sci. USA, 90: 10803-10808.

55.) Pappas, T., B. Gametchu and C. Watson (1995). Membrane estrogen receptors identified by multiple antibody labeling and impeded ligand binding. FASEB J., 9: 404-410.

56.) Pappas, T., B. Gametchu and C. Watson (1995). Membrane estrogen receptor-enriched GH3/B6 cells have an enhanced non-genomic response to estrogen. Endocrine, 3 : 743-749.

57.) Tesarik, J. and C. Mendoza (1995). Nongenomic effects of 17β-estradiol on maturing human oocytes. J. Clin. Endocrinol. Metabolism, 80: 1438-1443.

58.) Fiorelli, G., Gori, F., Frediani, U., Franceschelli, F., Tanini, A., Tosti-Guerra, C., Benvenuti, S., Gennari, L., Becherini, L. and Brandi, M.(1996). Membrane binding sites and non-genomic effects of estrogen in cultured human pre-osteoclastic cells. J. Steroid Biochem. Mol. Biol., 59:233-40.

59.) Watters J., Campbell J., Cunningham M., Krebs E. and Dorsa D. (1997). Rapid membrane effects of steroids in neuroblastoma cells: effects of estrogen on mitogen activated protein kinase signalling cascade and c-fos immediate early gene transcription. Endocrinology, 138 : 4030-3.

60.) Zheng, J. and Ramirez, V. (1997). Demonstration of membrane estrogen binding proteins in rat brain by ligand blotting using a 17β-estradiol-[125I]bovine serum albumin conjugate. J. Steroid Biochem. Molec. Biol., 62: 327-336.

61.) Razandi, M., Pedram, A., Greene, G. and Levin, E. (1999). Cell membrane and nuclear estrogen receptors (ERs) originate from a single transcript: studies of ERα and Erβ expressed in Chinese Hamster Ovary cells. Mol. Endocrinol., 13 : 307-319.

62.) Szego, C. M. and R. J. Pietras (1981). Membrane recognition and effector sites in steriod hormone action. In: Biochemical Actions of Hormones, Vol. VIII (G. Litwack, editor), Academic Press, New York, pp.307-464.

63.) Szego, C. M. and R. J. Pietras (1984). Lysosomal function in cellular activation: Propagation of the actions of hormones and other effectors. Int. Review of Cytology, 88: 1-246.

64.) Wehling, M. (1997). Specific, nongenomic actions of steroid hormones. Annu. Rev. Physiol., 59: 365-393.

65.) Nemere, I. and M. Farach-Carson (1998). Membrane receptors for steroid hormones. Biochem. Biophys. Res. Commun., 248: 443-449.

66.) Zhang, Q. -X., A. Borg and S. A. Fuqua (1993). An exon 5 deletion variant of the estrogen receptor frequently coexpressed with wild-type estrogen receptor in human breast cancer. Cancer Res., 53 :5882-5892.

67.) Chen, Z., Yu, L. and Chang, C. (1998). Stimulation of membrane-bound guanylate cyclase activity by 17-beta estradiol. Biochem. Biophys. Res. Commun., 252:639-42.

68.) Gu, Q., Korach, K. and Moss, R. (1999). Rapid action of 17beta-estradiol on kainate-induced currents in hippocampal neurons lacking intracellular estrogen receptors. Endocrinology, 140 :660-666.

69.) Kushner, P., Hort, E., Shine, J., Baxter, J. and Greene, G. (1990). Construction of cell lines that express high levels of the human estrogen receptor and are killed by estrogens. Mol. Endocrinol., 4: 1465-1473.

70.) Levenson, A. and Jordan, V. (1994). Transfection of human estrogen receptor (ER) cDNA into ER-negative mammalian cell lines. J. Steroid Biochem. Molec. Biol., 51: 229-239.

71.) Pietras, R. J. and C. M. Szego (1979). Estrogen receptors in uterine plasma membrane. J. Steroid Biochem. 11: 1471-1483.

72.) Otto, A. (1995). A one minute pulse of estradiol to MCF-7 breast cancer cells changes estrogen receptor binding properties and commits cells to induce estrogenic responses. J. Steroid Biochem. Molec. Biol., 54 :39-46.

73.) Chun, T. -Y., Gregg, D., Sarkar, D. and Gorsli, J. (1998). Differential regulation by estrogens of growth and prolactin synthesis in pituitary cells suggests that only a small pool of estrogen receptors is required for growth. Proc. Natl. Acad. Sci. USA, 95 : 2325-2330.

74.) Welshons W V; Grady L H; Judy B M; Jordan V C; Preziosi D E. (1993). Subcellular compartmentalization of MCF-7 estrogen receptor synthesis and degradation. Mol. Cell. Endocrinol., 94: 183-194.

75.) Pasic R; Djulbegovic B; Wittliff J L. (1990). Comparison of sex steroid receptor determinations in human breast cancer by enzyme immunoassay and radioligand binding. J. Clin. Lab. Anal., 4: 430-436.

76.) Paech, K., Webb, P., Kuiper, G., Nilsson, S., Gustafsson, J-A., Kushner, P. and Scanlan, T. (1997). Differential ligand activation of estrogen receptors ERα and ERβ at API sites. Science, 277: 1508-1510.

77.) Lewis G., Figari, I., Fendly, B., Wong, W., Carter, P., Gorman, C. and Shepard, H. (1993). Differential responses of human tumor cell lines to anti-p185HER2 monoclonal antibodies. Cancer Immunol. Immunother., 37: 255-263.

78.) Meyer, C., Schmid, R., Scriba, P. and Wehling, M. (1996). Purification and partial sequencing of high-affinity progesterone-binding sites(s) from porcine liver membranes. Eur. J. Biochem., 239: 726-731.

79.) Greene, G., Nolan, C., Engler, J. and Jensen, E. (1980). Monoclonal antibodies to human estrogen receptor. Proc. Natl. Acad. Sci. USA, 77: 5115-5119.

80.) Puca, G., Medici, N., Molinari, A., Moncharmont, B., Nola, E. and Sica, V. (1980). Estrogen receptor of calf uterus : An easy and fast purification procedure. J. Steroid Biochem., 12: 105-113.

81.) Holmes W., M. Sliwkowski, R. Akita, W. Henzel, J. Lee, J. Park, D. Yansura, N. Abadi, H. Raab, G. Lewis, H. Shepard, W. -J. Kuang, W. Wood, D. Goeddel and R. Vandlen (1992). Identification of heregulin, a specific activator of p185erbB2. Science, 256: 1205-1209.

82.) Green, S., Walter, P., Greene, G., Krust, A., Goffin, C., Jensen, E., Scrace, G., Waterfield, M. and Chambon, P. (1986). Cloning of the human oestrogen receptor cDNA. J. Steroid Biochem., 24: 77-83.

83.) Walter, P., Green, S., Greene, G., Krust, A., Bornert, J. -M., Jeltsch, J. -M., Staub, A., Jensen, E., Scrace, G., Waterfield, M. and Chambon, P. (1985). Cloning of the human estrogen receptor cDNA. Proc. Natl. Acad. Sci. USA, 82: 7889-7893.

84.) Xu, X. and Thomas, M. (1994). Estrogen receptor-mediated direct stimulation of colon cancer cell growth in vitro. Mol. Cell. Endocrinol., 105: 197-201.

85.) Pietras, R., Marquez, D., Chen, C., Li, D. and Lin, T. (1999). Antitumor effects of peptide antiestrogens that block estrogen receptor binding to steroid receptor coactivator-1 and to DNA in human breast cancer cells. Proc. Arn. Assoc. Cancer Res., 40: 637.

86.) Ignar-Trowbridge D. M., K. G. Nelson, M. C. Bidwell, S. W. Curtis, T. F. Washburn, J. A. McLachlan and K. S. Korach (1992). Coupling of dual signaling pathways: epidermal growth factor action involves the estrogen receptor. Proc. Natl. Acad. Sci. USA, 89: 4658-4666.

87.) Kato, S., H. Endoh, Y. Masuhiro, T. Kitamoto, S. Uchiyama, H. Sasaki, S. Masushige, Y. Gotoh, E. Nishida, H. Kawashima, D. Metzger and P. Chambon (1995). Activation of the estrogen receptor through phosphorylation by mitogen-activated protein kinase. Science, 270: 1491-1494.

88.) Smith C., O. Conneely and B. W. O'Malley (1995). Oestrogen receptor activation in the absence of ligand. Biochem. Soc. Trans., 935-939.

89.) Reddy, K., G. Mangold, A. Tandon, T. Yoneda, G. Mundy, A. Zilberstein and C. K. Osborne (1992). Inhibition of breast cancer cell growth in vitro by a tyrosine kinase inhibitor. Cancer Res., 52: 3636-3644.

90.) Sliwkowski M. X., G. Schaefer, R. W. Akita, J. A. Lofgren, V. D. Fitzpatrick, A. Nuijens, B. M. Fendly, R. A. Cerione, R. L. Vandlen and K. L. Carraway III (1994). Coexpression of erb B2 and erb B3 proteins reconstitutes a high affinity receptor for heregulin. J. Biol. Chem., 269: 14661-14669.

91.) Pegram, M., A. Lipton, R. Pietras, D. Hayes, B. Weber, J. Baselga, D. Tripathy, T. Twadell, J. Glaspy and D. Slamon (1995). Phase II study of intravenous recombinant humanized anti-p185 HER-2 monoclonal antibody (rhuMAb HER-2) plus cisplatin in patients with HER-2/neu overexpressing metastatic breast cancer. Proc.Am.Soc.Clin.Oncol., 14: 106.

92.) Bergeron, J J, Searle, N, Khan, M N and Posner, B I. (1986).

93.) Monje, P and Boland, R. (1999). Mol Cell Endocrinol, 147, 75-84

94.) Russell, K S, Haynes, M P, Sinha, D, Clerisme, E and Bender, J R. (2000). Proc Natl Acad Sci USA, 97, 5930-5935.

95.) Flouriot, G, Brand, H, Denger, S, Metivier, R, Kos, M, Reid, G, Sonntag-Buck, V and Gannon, F. (2000). EMBO J., 19, 4688-4700.

96.) Ahrnad, Singh, N and Glazer, R I. (1999). Biochem Pharmacol, 58, 425-430.

97.) Norfleet, A M, Clarke, C H, Gametchu, B and Watson, C S. (2000). FASEB J, 14, 157-165.

98.) Henderson, B E, Ross, R and Bernstein, L. (1988). Cancer Res, 48, 246-253.

99.) Kato, S, Endoh, H, Masuhiro, Y, Kitamoto, T, Uchiyama, S, Sasaki, H, Masushige, S, Gotoh, Y, Nishida, E, Kawashima, H, Daniel Metzger, and Pierre Chambon. (1995). Science, 270, 1491-1494.

100.) Weiler, P J and Wiebe, J P. (2000). Biochem Biophys Res Commun, 272, 731-737.

101.) Favata, M F, Horiuchi, K Y, Manos, E J, Daulerio, A J, Stradley, D A, Feeser, W S, Van Dyk, D E, Pitts, W J, Earl, R A, Hobbs, F, Copeland, R A, Magolda, R L, Scherle, P A and Trzaskos, J M. (1998). J Biol Chem, 273, 18623-18632.

102.) Haynes, M P, Sinha, D, Russell, K S, Collinge, M, Fulton, D, Morales-Ruiz, M, Sessa, W C and Bender, J R. (2000). Circ Res, 87, 677-682.

103.) Luconi, M, Muratori, M, Forti, G and Baldi, E. (1999). J Clin Endocrinol Metab, 84, 1670-1678.

104.) Levin, E R. (1999). Trends Endocrinol Metab, 10, 374-377

105.) Pietras, R J, Fendly, B M, Chazin, V R, Pegram, M, Howell, S B and Slamon, D J. (1994).Oncogene, 9, 1829-1838.

106.) Mendelsohn, M. E. (2000) Circulation Res. 87: 677.

1. A method of inhibiting the signalling of a membrane-associated estrogen receptor at the plasma membrane of a cell comprising contacting the membrane-associated estrogen receptor with an inhibitory ligand that binds to the ligand binding domain of the membrane-associated estrogen receptor thereby inhibiting membrane-associated estrogen receptor signalling.

2. The method of claim 1 wherein the inhibition of membrane-associated estrogen receptor signaling is measured by observing an inhibition in the estrogen-induced growth of MCF-7 breast cancer cells in vitro.

3. The method of claim 1 wherein the inhibition of membrane-associated estrogen receptor signaling is measured by observing an inhibition in the growth of MCF-7 breast cancer xenografts in nude mice.

4. The method of claim 1, wherein the inhibitory ligand does not cross the cell membrane.

5. The method of claim 4, wherein the inhibitory ligand is selected from the group consisting of monoclonal antibodies Ab 1 and Ab 2.

6. The method of claims 1 further comprising contacting the cell with an anti-Her-2 immunoglobulin polypeptide under conditions which allow the anti-Her-2 immunoglobulin polypeptide to bind to Her-2 on the surfaces of the cancer cell to a degree sufficient to inhibit the growth of the cancer cell.

7. The method of claim 1, wherein the cell is a breast cancer cell.

8. The method of claim 1, wherein the cell is an ovarian cancer cell.

9. The method of claim 1 further comprising treating the cell with a conventional cancer therapy selected from the group consisting of surgical excision and chemotherapy.

10. A method of inhibiting the growth of a breast cancer cell having an estrogen receptor associated with its cell membrane comprising contacting the cell with an amount of anti-estrogen receptor immunoglobulin polypeptide sufficient to inhibit cell growth, wherein the anti-estrogen receptor immunoglobulin polypeptide recognizes and binds a ligand binding domain in the estrogen receptor.

11. A method of inhibiting the growth of a breast lineage cell having disregulated cell growth comprising the steps of:
a) confirming the presence of an estrogen receptor associated with the membrane of the breast lineage cell;
b) providing a estrogen receptor immunoglobulin polypeptide specific for an epitope within the ligand binding domain of the estrogen receptor, the anti-estrogen receptor immunoglobulin polypeptide being selected to produce inhibition of breast cell growth; and
c) contacting the cell with the anti-estrogen receptor immunoglobulin polypeptide under conditions which allow the anti-estrogen receptor immunoglobulin polypeptide to interact with the estrogen receptor associated with the surface membranes of the breast lineage cell to a degree sufficient to inhibit the growth of the breast cancer cell.

12. The method of claim 11 wherein the anti-estrogen receptor immunoglobulin polypeptide is selected from the group consisting of monoclonal antibodies Ab 1 and Ab 2.

13. The method of claim 11 further comprising contacting the cell with an anti-HER-2 immunoglobulin polypeptide under conditions which allow the anti-HER-2 immunoglobulin polypeptide to bind to HER-2 on the surfaces of the cancer cell to a degree sufficient to inhibit the growth of the cancer cell.

14. The method of claim 13 further comprising treating the cancer cell with an anti-HER-1/EGF receptor immunoglobulin polypeptide or with a conventional therapy selected from the group consisting of surgical excision and chemotherapy.

15. A method of radioimaging breast cancer cells which express membrane-associated estrogen receptor comprising the steps of first administering to an individual suspected of having breast cancer, a pharmaceutical composition that comprises a pharmaceutically acceptable carrier or diluent, and conjugated compound that comprises an anti-estrogen receptor immunoglobulin polypeptide and a radioactive active moiety wherein the conjugated compound is present in an amount effective for diagnostic use in humans suffering from breast cancer and then detecting the localization and accumulation of radioactivity in the individual's body.

16. A kit for use in methods for inhibiting the growth of breast lineage cells which express an estrogen receptor comprising a container, a composition contained within the container, wherein the composition includes an anti-estrogen receptor immunoglobulin polypeptide and instructions for using the anti-estrogen receptor immunoglobulin polypeptide in vivo or in vitro.

17. A method of inhibiting the signalling of a membrane-associated estrogen receptor at the plasma membrane of a cell comprising contacting the membrane-associated estrogen receptor with steroidal and non-steroidal inhibitory compounds that selectively bind to the membrane-associated estrogen receptor, thereby inhibiting membrane-associated estrogen receptor signalling.

18. A method of inhibiting the signalling of a membrane-associated estrogen receptor at the plasma membrane of a cell comprising selective blockade of a component of the intracellular signal transduction pathway that emanate from activation of the membrane-associated estrogen receptor, including MAP kinase, PI3K/Akt kinase, erb B tyrosine kinase, adenylate cyclase, G-protein-coupled, calcium homeostasis and nitric oxide-based signal transduction pathways, thereby inhibiting membrane-associated estrogen receptor signalling.
